(19) United States
(12) Patent Application Publication
(10) Pub. No.: US 2023/0277757 A1
(43) Pub. Date: Sep. 7, 2023

(54) POINT OF CARE DRUG DELIVERY APPARATUS AND METHOD

(71) Applicant: Genentech, Inc., South San Francisco, CA (US)

(72) Inventors: Geoffrey Colin Wise, San Jose, CA (US); Kai Zheng, Foster City, CA (US); Bryan Andrew Click, San Francisco, CA (US); Stefan Thomas Yohe, San Francisco, CA (US); Lorenzo Myles Paredes Pesino, San Francisco, CA (US); Hemanth Kumar Amarchinta, San Jose, CA (US); Adam Daniel Farchone, San Jose, CA (US)

(21) Appl. No.: 18/173,673

(22) Filed: Feb. 23, 2023

Related U.S. Application Data
(63) Continuation of application No. PCT/US2021/047951, filed on Aug. 27, 2021.
(60) Provisional application No. 63/235,060, filed on Aug. 19, 2021.
(60) Provisional application No. 63/071,901, filed on Aug. 28, 2020.

(51) Int. Cl. A61M 5/14; A61M 5/162

(52) U.S. Cl. CPC A61M 5/1408; A61M 5/162; A61M 5/1409

(57) ABSTRACT

An apparatus (100) and a method for a point of care drug product delivery are provided. A point of care drug product delivery apparatus includes a central interface member (110) that serves as an interface between a saline source, a drug product source, and an infusion line. The central interface member facilitates priming the infusion line with saline from the saline source, infusing drug product to a patient from the drug product source, and flushing the infusion line with saline from the saline source. According to aspects of the current subject matter, the central interface member provides and/or forms fluidic connections and/or passageways between various ones of the saline source, the drug product source, and the infusion line.

### CROSS-REFERENCE TO RELATED APPLICATIONS

The present application claims priority to U.S. Provisional Application No. 63/235,060, filed on Aug. 19, 2021, and titled “Point of Care Drug Delivery Apparatus and Method,” and U.S. Provisional Application No. 63/071,901, filed on Aug. 28, 2020, and titled “Point of Care Drug Delivery Apparatus and Method,” the entirety of each of which is incorporated by reference herein.

### TECHNICAL FIELD

The current subject matter described herein relates generally to drug product delivery to a patient. More particularly, the current subject matter relates to an apparatus and a method that provides for point of care drug product delivery to a patient.

### BACKGROUND

Infusion of a drug product, such as one or more pharmaceuticals, biopharmaceuticals, and/or biologics, to a patient may involve an intravenous administration of the drug product into the patient. One or more health care providers may be responsible for the intravenous administration, which may include, for example, dose preparation procedures and patient preparation procedures to provide for the drug to be intravenously administered to the patient.

### SUMMARY

Aspects of the current subject matter relate to a point of care drug product delivery apparatus and method. The point of care drug product delivery apparatus and method, consistent with implementations of the current subject matter, results in a reduction of time and steps as well as provides a simplified process for drug product delivery to a patient as compared to conventional methods.

The point of care drug product delivery apparatus and method, according to aspects of the current subject matter, provides for a central component to interface with a saline source, a drug product source, and an infusion line, for priming the infusion line with saline, infusing drug product to a patient, and flushing the infusion line with saline.

The point of care drug product delivery apparatus and method simplifies a health care provider's workflow by reducing preparation and infusion steps by allowing intravenous (IV) infusion of a liquid drug product from its primary container. It does not require dilution into IV bags prior to administration, and it eliminates the need to switch IV bags between priming, dose administration, and flushing. Therefore, it provides a more convenient and faster IV administration option for healthcare systems while improving patient experience. The point of care drug product delivery apparatus and method improves safety by being a closed system. It eliminates the needs for a closed system drug transfer device and reduces additional supplies, such as saline bags and secondary intravenous sets that may typically be required.

According to aspects disclosed herein, an apparatus is provided. The apparatus includes a central interface member including a cavity surrounded by an outer wall, a plurality of access points formed through respective surfaces of the outer wall; an infusion port including a first end and a second end, the first end of the infusion port coupled to a first access point of the plurality of access points; a saline port including a first end and a second end, the second end of the saline port coupled to a second access point of the plurality of access points, such that a first passageway is formed between the saline port and the infusion port through the cavity of the central interface member; and a drug product port including a first end and a second end, the second end of the drug product port coupled to a third access point of the plurality of access points, such that a second passageway is formed between the drug product port and the infusion port through the cavity of the central interface member.

In another, interrelated aspect, a method is provided. The method includes priming, via a saline port coupled to an infusion port by a first passageway formed in a cavity of a central interface member to which the saline port and the infusion port are coupled, a first quantity of saline; infusing, via a drug product port coupled to the infusion port by a second passageway formed in the cavity of the central interface member to which the drug product port is coupled, an infusion volume of the drug product into the patient; and infusing, via at least the saline port and the infusion port, a second quantity of saline into the patient.

In another, interrelated aspect, an apparatus is provided. The apparatus includes a connection component and a port manifold. The connection component includes a central connection member configured to connect to an infusion stand; and a first vial connection member coupled to a first support arm, the first support arm extending from the central connection member, the first vial connection member configured to support a first vial adaptor. The port manifold includes a port configured to be inserted into a saline source; an infusion line configured to provide a passageway between the port and an intravenous administration set; and a first drug product line configured to connect to the first vial adaptor at a first end thereof, the first drug product line coupled to the infusion line at a second end thereof.

In another, interrelated aspect, a method is provided. The method includes priming, via an infusion line coupled to a saline source, a first quantity of saline to prime the infusion line; infusing, from a first vial and via a drug product line coupled to the infusion line, an infusion volume of the drug product into the patient; and flushing, via the infusion line coupled to the saline source, into the patient a second quantity of saline.

In another, interrelated aspect, an apparatus is provided. The apparatus includes a central interface member, an infusion port, and a fluid port. The central interface member may include a cavity surrounded by an outer wall and a plurality of access points formed through respective surfaces of the outer wall. The infusion port may be coupled to a first access point of the plurality of access points. The infusion port may be coupled to tubing. The fluid port may be coupled to a second access point of the plurality of access points. The fluid port may be positioned opposite the infusion port such that a first passageway is formed between the fluid port and the infusion port through the cavity of the central interface member. The fluid port may be coupled to a drug product source. The infusion port, the fluid port, the first access point, and the second access point may be aligned along a central longitudinal axis of the central interface member.

In another, interrelated aspect, an apparatus is provided. The apparatus includes a central interface member, an infusion port, and a fluid port. The central interface member may include a cavity surrounded by an outer wall and a plurality of access points formed through respective surfaces of the outer wall. The infusion port may be coupled to a first access point of the plurality of access points. The infusion port may be coupled to an intravenous administration set. The fluid port may be positioned opposite the infusion port such that a first passageway is formed between the fluid port and the infusion port through the cavity of the central interface member. The fluid port may be non-contemporaneously coupled to a saline source and a drug product source.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings:

FIG. 1A-FIG. 1B illustrate aspects of a point of care drug product delivery apparatus and method consistent with implementations of the current subject matter;

FIG. 2A-FIG. 2F illustrate aspects of a point of care drug product delivery apparatus and method consistent with additional implementations of the current subject matter;

FIG. 3A-FIG. 3D illustrate aspects of a point of care drug product delivery apparatus and method consistent with additional implementations of the current subject matter;

FIG. 4A-FIG. 4D illustrate aspects of a point of care drug product delivery apparatus and method consistent with additional implementations of the current subject matter;

FIG. 5A-FIG. 5D illustrate aspects of a point of care drug product delivery apparatus and method consistent with additional implementations of the current subject matter;

FIG. 6A-FIG. 6D illustrate aspects of a point of care drug product delivery apparatus and method consistent with additional implementations of the current subject matter;

FIG. 7 illustrates aspects of a point of care drug product delivery apparatus consistent with additional implementations of the current subject matter; and

FIG. 8A-FIG. 8C are diagrams illustrating aspects of a point of care drug product delivery apparatus and method consistent with additional implementations of the current subject matter.

FIG. 9A-FIG. 9G illustrate aspects of a point of care drug product delivery apparatus and method consistent with additional implementations of the current subject matter;

FIG. 10 illustrates a graph depicting concentration kinetics of a drug product delivered using a point of care drug product delivery apparatus and method consistent with additional implementations of the current subject matter;

FIG. 11A-FIG. 11F illustrate aspects of a point of care drug product delivery apparatus and method consistent with additional implementations of the current subject matter;

FIG. 12A-FIG. 12C illustrate aspects of a point of care drug product delivery apparatus and method consistent with additional implementations of the current subject matter; and

FIG. 13A-FIG. 13C illustrate aspects of a point of care drug product delivery apparatus and method consistent with additional implementations of the current subject matter;

When practical, similar reference numbers denote similar structures, features, or elements.

### DETAILED DESCRIPTION

### I. Definitions

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, cats, monkeys, goat, sheep, cows, deer, and other non-mammalian animals. In some embodiments, a patient is human.

Dosages may be varied depending upon the requirements of the patient and the compound being employed. The dose administered to a patient, in the context of the present disclosure, should be sufficient to elicit a beneficial therapeutic response in the patient over time. The size of the dose also will be determined by the existence, nature, and extent of any adverse side-effects. Determination of the proper dosage for a particular situation is within the skill of the practitioner. Dosage amounts and intervals can be adjusted individually to provide levels of the administered compound effective for the particular clinical indication being treated. This will provide a therapeutic regimen that is commensurate with the severity of the individual's disease state.

### II. Pharmaceutical Compositions

The term “drug product” is used in accordance with its plain ordinary meaning and refers to any pharmaceutical composition or formulation. The drug product may be a drug for treatment and/or prevention of any disease. Preferably, the drug product is an aqueous composition, or is diluted with an aqueous composition prior to administration to a patient. The drug product may be, without limitation, an anticancer agent, an anti-inflammatory agent, a biologic agent, a peptide, a small molecule, a nucleic acid, a lipid, and the like.

An “anticancer agent” as used herein refers to a molecule (e.g. compound, peptide, protein, nucleic acid, 0103) used to treat cancer through destruction or inhibition of cancer cells or tissues. Anticancer agents may be selective for certain cancers or certain tissues. In embodiments, anticancer agents herein may include epigenetic inhibitors and multi-kinase inhibitors. “Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is a biologic. In some embodiments, an anti-cancer agent is an immunotherapy agent. In some embodiments, an anti-cancer agent is an immune checkpoint inhibitor. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec.RTM.), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; anti sense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenyl acetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol.™ (i.e. paclitaxel), Taxotere.™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, Inanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹TH ⁹⁰Y or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like. In an embodiment, the anti-cancer agent is an immune checkpoint inhibitor (e.g., atezolizumab (Tecentriq®), pembrolizumab (Keytruda®), Ipilimumab, Nivolumab (Opdivo®), Avelumab, Durvalumab, Cemiplimab, or spartalizumab).

### III. Methods of Use

As used herein, the term “administering” generally means intravenous administration, unless otherwise indicated. Other modes of administration include, without limitation: administration as a suppository, topical contact, oral, parenteral, intraperitoneal, intramuscular, intralesional, intrathecal, intranasal, subcutaneous administration, implantation of a slow-release device, e.g., a mini-osmotic pump, transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, transdermal patches, etc.

It is understood that the examples and embodiments described herein are for illustrative purposes only and that various modifications or changes in light thereof will be suggested to persons skilled in the art and are to be included within the spirit and purview of this application and scope of the appended claims.

### IV. Description

The current subject matter is directed to a point of care drug product delivery apparatus and method. The point of care drug product delivery apparatus and method, consistent with implementations of the current subject matter, results in a reduction of time and steps as well as provides a simplified process for drug product delivery to a patient as compared to conventional methods.

The point of care drug product delivery apparatus and method, according to aspects of the current subject matter, incorporates a central component to interface with a saline source, a drug product source, and an infusion line, for priming the infusion line with saline, infusing drug product to a patient, and flushing the infusion line with saline.

The point of care drug product delivery apparatus and method, consistent with implementations of the current subject matter, simplifies a health care provider's workflow by reducing preparation and infusion steps by allowing intravenous (IV) infusion of a drug product from its primary container. Delivering the drug product to the patient using the point of care drug product delivery apparatus and method does not require dilution into IV bags prior to administration, and eliminates the need to switch IV bags between priming, dose administration, and flushing. Therefore, the point of care drug product delivery apparatus and method provides a more convenient and faster IV administration option for healthcare systems while improving patient experience. The point of care drug product delivery apparatus and method improves safety by being a closed system. It eliminates the needs for a closed system drug transfer device and reduces additional supplies, such as saline bags and secondary intravenous sets that may typically be required.

For a conventional drug product infusion procedure, one or more health care providers may be responsible for the intravenous administration, which may include, for example, dose preparation procedures and patient preparation procedures to provide for the drug product to be intravenously directed to the patient. For example, the dose preparation procedures may involve one or more health care providers preparing a diluted drug product in an intravenous bag, which expends valuable resources of time (e.g., the time of the health care providers to prepare the diluted drug product) and materials (e.g., the intravenous bag). Moreover, the dose preparation procedures need to be conducted in a sterile environment, further expending resources including sterile equipment (e.g., health care provider gloves and masks) and hospital or clinical space. Additionally, such dose preparation procedures inherently are at risk for error due to the reliance on human interaction in the preparation of the diluted drug product.

The point of care drug product delivery apparatus and method consistent with implementations of the current subject matter simplifies the conventional drug product infusion procedure by providing for the drug product to be infused to the patient from the vial or container. This decreases the time, material, and space resources that are involved in conventional dose preparation procedures.

According to aspects of the current subject matter, a point of care drug product delivery apparatus (also referred to herein as a drug product delivery apparatus) includes a central interface member that serves as an interface between a saline source, a drug product source, and an infusion line (e.g., an IV administration set). The IV administration set is configured to administer fluids to a patient. For example, the IV administration set may interface with an infusion pump, which operates to pump the fluid contained in the IV administration set at a prescribed rate. At a distal end of the infusion line is an IV catheter or a needle that is inserted into the patient for delivering the fluid from the infusion line to the patient. The central interface member facilitates priming the infusion line with saline from the saline source, infusing drug product to the patient from the drug product source, and flushing the infusion line with saline from the saline source.

According to aspects of the current subject matter, the central interface member provides and/or forms fluidic connections and/or passageways between various ones of the saline source, the drug product source, and the infusion line (e.g., the IV administration set), as further described herein.

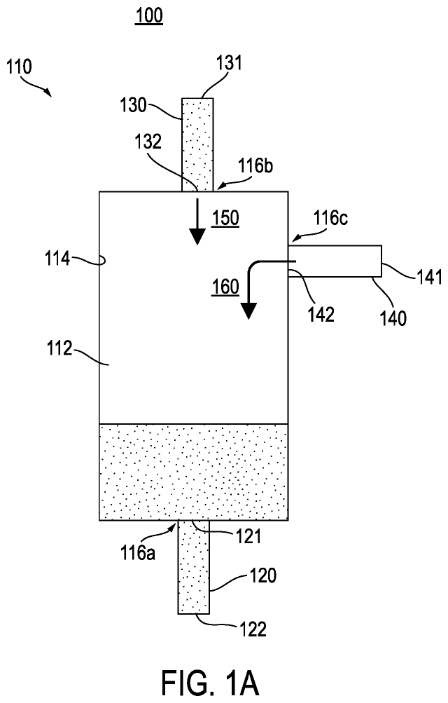

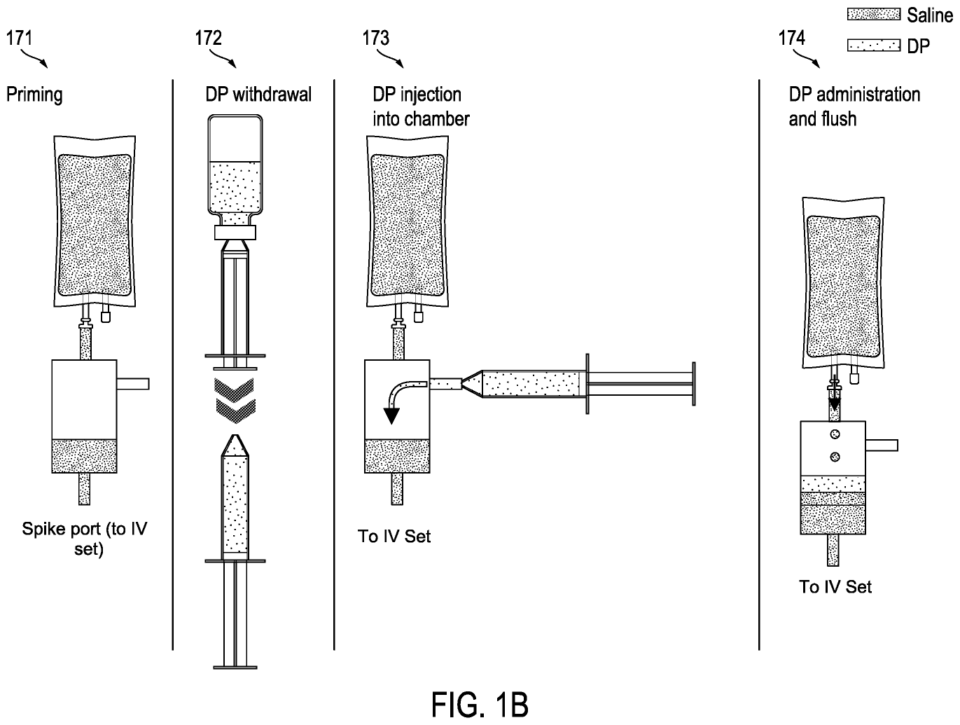

FIGS. 1A-13C illustrate various examples of a drug product delivery apparatus 100, 200, 300, 400, 700, 800, 900, 1100, 1300 consistent with implementations of the current subject matter. The features, properties, and/or components of each of the drug product delivery apparatuses 100, 200, 300, 400, 700, 800, 900, 1100, and 1300 described herein may be implemented on one or more of the other disclosed drug product delivery apparatuses 100, 200, 300, 400, 700, 800, 900, 1100, and 1300. For example, the features, properties, and/or components of the drug product delivery apparatus 100 may be implemented in the drug product delivery apparatus 200, 300, 400, 700, 800, 900, 1100, and/or 1300. The features, properties, and/or components of the drug product delivery apparatus 200 may be implemented in the drug product delivery apparatus 100, 300, 400, 700, 800, 900, 1100, and/or 1300. The features, properties, and/or components of the drug product delivery apparatus 300 may be implemented in the drug product delivery apparatus 100, 200, 400, 700, 800, 900, 1100, and/or 1300. The features, properties, and/or components of the drug product delivery apparatus 400 may be implemented in the drug product delivery apparatus 100, 200, 300, 700, 800, 900, 1100, and/or 1300. The features, properties, and/or components of the drug product delivery apparatus 700 may be implemented in the drug product delivery apparatus 100, 200, 300, 400, 800, 900, 1100, and/or 1300. The features, properties, and/or components of the drug product delivery apparatus 800 may be implemented in the drug product delivery apparatus 100, 200, 300, 400, 700, 900, 1100, and/or 1300. The features, properties, and/or components of the drug product delivery apparatus 900 may be implemented in the drug product delivery apparatus 100, 200, 300, 400, 700, 800, 1100, and/or 1300. The features, properties, and/or components of the drug product delivery apparatus 1100 may be implemented in the drug product delivery apparatus 100, 200, 300, 400, 700, 800, 900, and/or 1300. The features, properties, and/or components of the drug product delivery apparatus 1300 may be implemented in the drug product delivery apparatus 100, 200, 300, 400, 700, 800, 900, and/or 1100.

FIG. 1A illustrates aspects of a drug product delivery apparatus 100 consistent with implementations of the current subject matter. The drug product delivery apparatus 100 includes a central interface member 110. The central interface member 110 includes a cavity 112 surrounded by an outer wall 114 and a plurality of access points 116 formed through respective surfaces of the outer wall 114. Shown in FIG. 1A are three access points: a first access point 116a, a second access point 116b, and a third access point 116c.

The drug product delivery apparatus 100 also includes an infusion port 120, a saline port 130, and a drug product port 140. According to aspects of the current subject matter, each of the ports 120, 130, and 140 has a first end and a second end, and each of the ports 120, 130, and 140 is coupled at one end to a respective access point 116a, 116b, and 116c of the central interface member 110.

The infusion port 120 has a first end 121, coupled to the first access point 116a of the central interface member 110, and a second end 122. The second end 122 of the infusion port 120 may be configured to connect to an IV administration set for delivering fluid from the infusion port 120.

The saline port 130 has a first end 131 and a second end 132. The second end 132 of the saline port 130 is coupled to the second access point 116b of the central interface member 110. A first passageway 150 is thus formed between the saline port 130 and the infusion port 120 by way of the second access point 116b and the first access point 116a. The first passageway 150 is a fluidic connection through the cavity 112 of the central interface member 110. The first end 131 of the saline port 130 is configured to be coupled to a saline source, thus allowing the saline port 130 to deliver saline from the saline source through the cavity 112 of the central interface member 110 (e.g., through the first passageway 150) to the infusion port 120. In some implementations, the saline port 130 is terminated with a spike for coupling to the saline source (e.g., a saline bag or the like).

The drug product port 140 of the drug product delivery apparatus 100 has a first end 141 and a second end 142. The second end 142 of the drug product port 140 is coupled to the third access point 116c of the central interface member 110. A second passageway 160 is thus formed between the drug product port 140 and the infusion port 120 by way of the third access point 116c and the first access point 116a. The second passageway 160 is a fluidic connection through the cavity 112 of the central interface member 110. The first end 141 of the drug product port 140 is configured to be coupled to a drug product source, thus allowing the drug product port 140 to deliver drug product from the drug product source through the cavity 112 of the central interface member 110 (e.g., through the second passageway 160) to the infusion port 120. The drug product source may be, for example, a syringe. The drug product source may be, for example, a vial or a container. In one implementation, the first end 141 of the drug product port 140 may be, or may be engaged with, a vial adaptor configured to engage with the vial such that the drug product flows from the vial and the vial adaptor, through the drug product port 140, into the cavity 112 of the central interface member 110.

As shown in FIG. 1A, the drug product delivery apparatus 100, by way of the central interface member 110 and the access points 116a, 116b, and 116c, provides for fluid connections to be established between the saline port 130 and the infusion port 120 (e.g., the first passageway 150) for priming an IV administration set with saline and flushing the IV administration set with saline from a saline source, and between the drug product port 140 and the infusion port 120 (e.g., the second passageway 160) for delivering drug product from a drug product source to a patient via the IV administration set.

FIG. 1B illustrate aspects of operation of the drug product delivery apparatus 100, consistent with implementations of the current subject matter. Drug product delivery utilizing the drug product delivery apparatus 100 includes, according to some implementations, four stages, as shown in FIG. 1B: priming 171, drug product withdrawal 172, drug product injection 173, and drug product administration and flushing 174. Each of the stages is accomplished via pumping from an infusion pump that interfaces with the IV administration set coupled to the infusion port 120.

The priming stage 171 involves infusing, via the saline port 130, a first quantity of saline into the cavity 112 of the central interface member 110 from a saline source. For example, the saline port 130 may be terminated with a spike or the like for coupling to the saline source, such as a saline bag. The priming provides for the first quantity of saline to be distributed to the cavity 112 and the infusion port 120 through the first passageway 150.

The drug product withdrawal 172, consistent with implementations of the current subject matter, may include withdrawing, with a syringe or the like, a quantity (e.g., an infusion volume) of the drug product from the drug product source, such as a vial or container.

The drug product injection 173 includes injecting the infusion volume of the drug product, contained in the syringe, for example, via the drug product port 140 of the central interface member 110. For example, the infusion volume may be injected through the drug product port 140 to the cavity 112.

The drug product administration and flushing 174 includes, according to aspects of the current subject matter, infusing the infusion volume of the drug product and a second quantity of saline into the patient via the infusion port 120 and the IV administration set. For example, after the priming 171 of the first quantity of saline and the drug product injection 173 of the infusion volume, the infusion volume and the second quantity of saline may be flushed through the infusion port 120 to the IV administration set. The infusion of the second quantity of saline may ensure that the infusion volume of the drug product is delivered to the patient.

According to additional aspects of the current subject matter, movable conduits may be provided internal to the cavity of the central interface member. The movable conduits may be tubes, pipes, or the like positioned within the cavity that interface with the access points such that secure passageways are formed between various ones of the access points, and thus between various ones of the saline port, the infusion port, the drug product port, and a flush port (if incorporated as further described herein). The movable conduits thus provide for passageways (e.g., fluidic connections) to allow for the priming and flushing of saline and the delivery of drug product. According to aspects of the current subject matter, the central interface member including movable conduits is a type of valve for providing fluidic connections between the various access points, as further described herein.

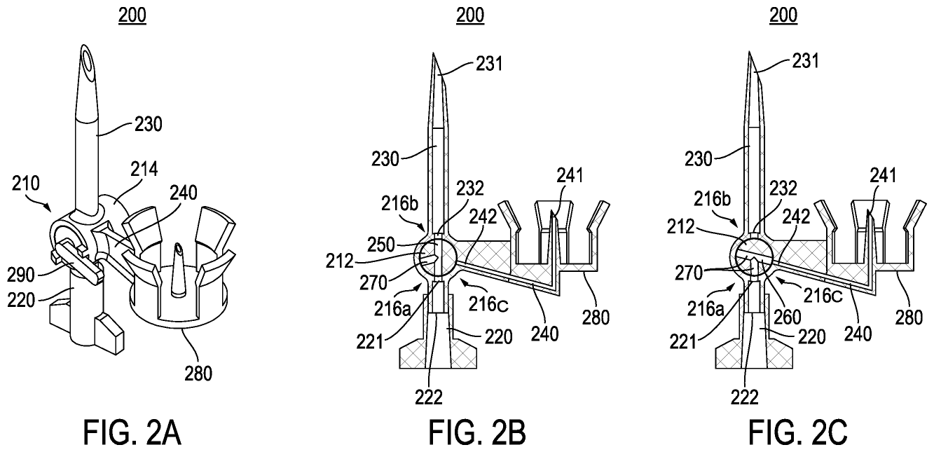

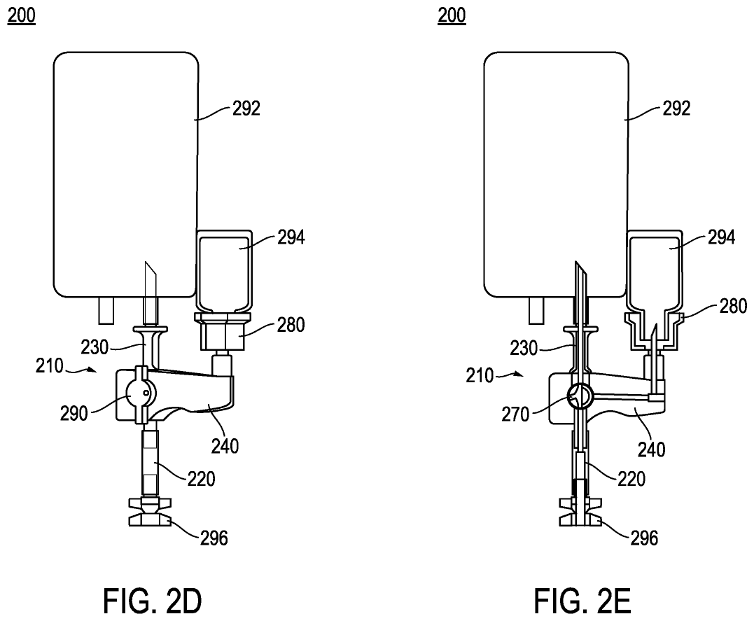

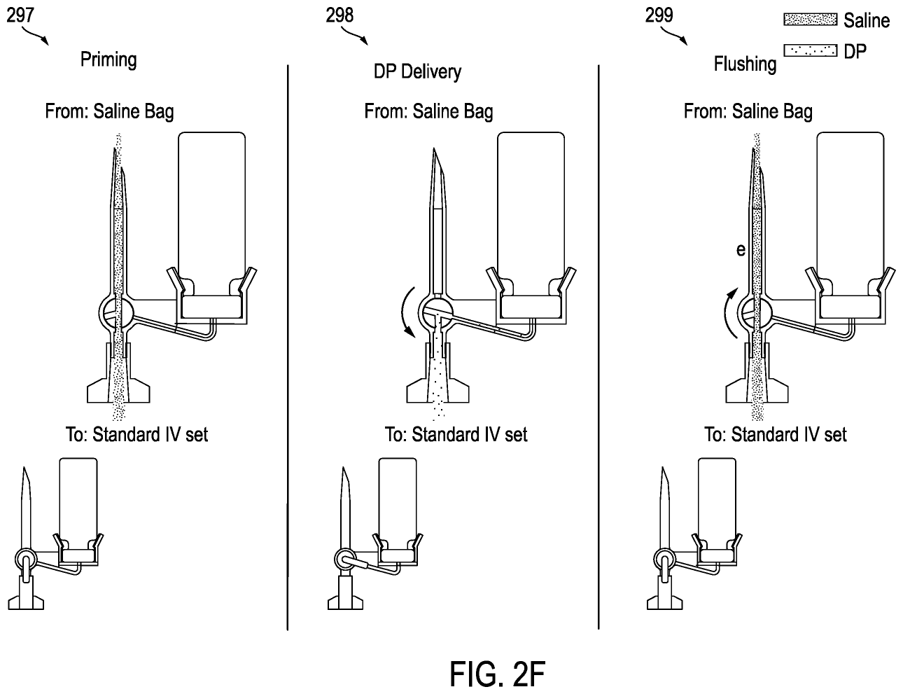

FIG. 2A-FIG. 2C illustrate aspects of a drug product delivery apparatus 200 consistent with implementations of the current subject matter. FIG. 2A is a perspective view and FIG. 2B and FIG. 2C are cross-sectional views of the drug product delivery apparatus 200.

The drug product delivery apparatus 200 includes a central interface member 210. The central interface member 210 includes a cavity 212 surrounded by an outer wall 214 and a plurality of access points 216 formed through respective surfaces of the outer wall 214. Shown in FIG. 2B and FIG. 2C are three access points: a first access point 216a, a second access point 216b, and a third access point 216c.

The drug product delivery apparatus 200 includes movable conduits 270, as shown in the cross-sectional views of FIG. 2B and FIG. 2C. The movable conduits 270 are positioned within the cavity 212 and interface with the plurality of access points 216 as further described herein.

The drug product delivery apparatus 200 also includes an infusion port 220, a saline port 230, and a drug product port 240. According to aspects of the current subject matter, each of the ports 220, 230, and 240 has a first end and a second end, and each of the ports 220, 230, and 240 is coupled at one end to a respective access point 216a, 216b, and 216c of the central interface member 210.

The infusion port 220 has a first end 221, coupled to the first access point 216a of the central interface member 210, and a second end 222. The second end 222 of the infusion port 220 may be configured to connect to an IV administration set for delivering fluid from the infusion port 220.

The saline port 230 has a first end 231 and a second end 232. The second end 232 of the saline port 230 is coupled to the second access point 216b of the central interface member 210.

Consistent with implementations of the current subject matter, the movable conduits 270 are configured to move between at least a first position and a second position. In the first position, the movable conduits 270 interface with the plurality of access points 216 such that a first passageway 250 is defined between the second access point 216b and the first access point 216a to provide fluid connection between the saline port 230 and the infusion port 220.

The first end 231 of the saline port 230 is configured to be coupled to a saline source, thus allowing the saline port 230 to deliver saline from the saline source through the first passageway 250 formed by the movable conduits 270 to the infusion port 220. In some implementations, the saline port 230 is terminated with a spike for coupling to the saline source (e.g., a saline bag or the like).

The drug product port 240 of the drug product delivery apparatus 200 has a first end 241 and a second end 242. The second end 242 of the drug product port 240 is coupled to the third access point 216c of the central interface member 210.

Consistent with implementations of the current subject matter, in the second position, the movable conduits 270 interface with the plurality of access points 216 such that a second passageway 260 is defined between the third access point 216c and the first access point 216a to provide fluid connection between the drug product port 240 and the infusion port 220.

The first end 241 of the drug product port 240 is configured to be coupled to a drug product source, thus allowing the drug product port 240 to deliver drug product from the drug product source through the second passageway 260 to the infusion port 220.

As shown in FIG. 2A-FIG. 2C, the first end 241 of the drug product port 240 may be, or may be engaged with, a vial adaptor 280 configured to engage with a vial (not shown in FIG. 2A-FIG. 2C) such that the drug product flows from the vial and the vial adaptor 280, through the drug product port 240 and the second passageway 260 to the infusion port 220.

Consistent with implementations of the current subject matter, the drug product delivery apparatus 200 may include a control member 290, as shown in FIG. 2A. The control member 290 is coupled to the movable conduits 270 to move the movable conduits 270 between the first position and the second position. For example, the control member 290 may be configured such that movement, such as rotation, of the control member 290 causes the movable conduits 270 to rotate within the cavity 212 of the central interface member 210. Thus, movement of the control member 290 causes the movable conduits 270 to be aligned to form the first passageway 250 and the second passageway 260.

According to aspects of the current subject matter, the first position of the movable conduits and the second position of the movable conduits 270 do not concurrently occur. That is, while the first passageway 250 is formed and open between the second access point 216b and the first access point 216a to provide fluid connection between the saline port 230 and the infusion port 220, the second passageway 260 is not formed (e.g., the movable conduits 270 are positioned in such a way that there is no fluid connection between the drug product port 240 and the infusion port 220). When the second passageway 260 is formed and open between the third access point 216c and the first access point 216a to provide fluid connection between the drug product port 240 and the infusion port 220, the first passageway 250 is not formed (e.g., the movable conduits 270 are positioned in such a way that there is no fluid connection between the saline port 230 and the infusion port 220). This arrangement allows for saline priming and flushing to occur between the saline port 230 and the infusion port 220 separate from delivery of the drug product from the drug product port 240 to the infusion port 220.

FIG. 2D and FIG. 2E illustrate additional aspects of the drug product delivery apparatus 200 consistent with implementations of the current subject matter. FIG. 2D is a perspective view and FIG. 2E a cross-sectional view of the drug product delivery apparatus 200 coupled to a saline source 292 at the saline port 230 and coupled to a vial 294 at the drug product port 240. The infusion port 220 is coupled to an IV administration set 296. As shown in the cross-sectional view of FIG. 2E, the movable conduits 270 are arranged such that the first passageway 250 is opened between the saline port 230 and the infusion port 220. In the configuration shown in FIG. 2E, the second passageway 260 is not formed (e.g., the movable conduits 270 are positioned in such a way that there is no fluid connection between the drug product port 240 and the infusion port 220).

FIG. 2F illustrate aspects of operation of the drug product delivery apparatus 200, consistent with implementations of the current subject matter. Drug product delivery utilizing the drug product delivery apparatus 200 includes, according to some implementations, three stages, as shown in FIG. 2F: priming 297, drug product delivery 298, and flushing 299. The drug product delivery apparatus 200 is shown in cross-sectional views to illustrate aspects of the movable conduits 270, the first passageway 250, and the second passageway 260. Front views are also provided to illustrate a corresponding position of the control member 290. Each of the stages is accomplished via pumping from an infusion pump that interfaces with the IV administration set coupled to the infusion port 220.

In the priming stage 297, the movable conduits 270 are positioned in the first position such that the first passageway 250 is formed between the saline port 230 and the infusion port 220, allowing saline to flow from the saline source (e.g., a saline bag), through the first passageway 250, to the infusion port 220 (coupled to an IV administration set). The priming 297 involves infusing, via the saline port 230, a first quantity of saline into the drug product delivery apparatus 200. The saline port 230 may be terminated with a spike or the like for coupling to the saline source, such as the saline bag. The priming provides for the first quantity of saline to be distributed to the infusion port 220 through the first passageway 250.

For the stage of the drug product delivery 298, the movable conduits 270 are moved into the second position. For example, the control member 290 is rotated such that the second passageway 260 is formed between the drug product port 240 and the infusion port 220. This allows drug product to flow from the vial to the drug product port 240, through the second passageway 260, to the infusion port 220 (coupled to the IV administration set).

After the drug product delivery 298, the flushing 299 is the next stage of operation of the drug product delivery apparatus 200. The movable conduits 270 are returned to the first position such that the first passageway 250 is formed. A second quantity of saline is flushed through the first passageway 250 to the infusion port 220. The infusion of the second quantity of saline may ensure that the infusion volume of the drug product is delivered to the patient.

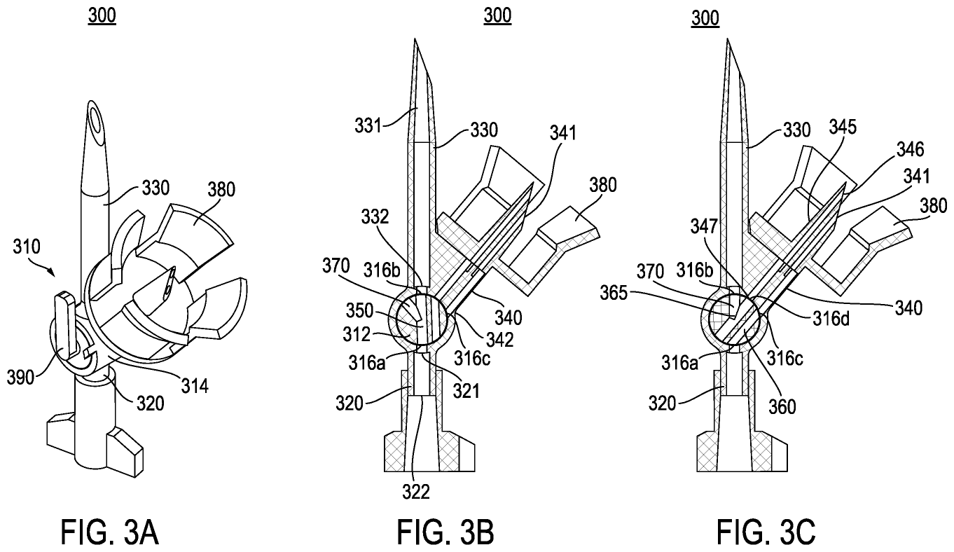

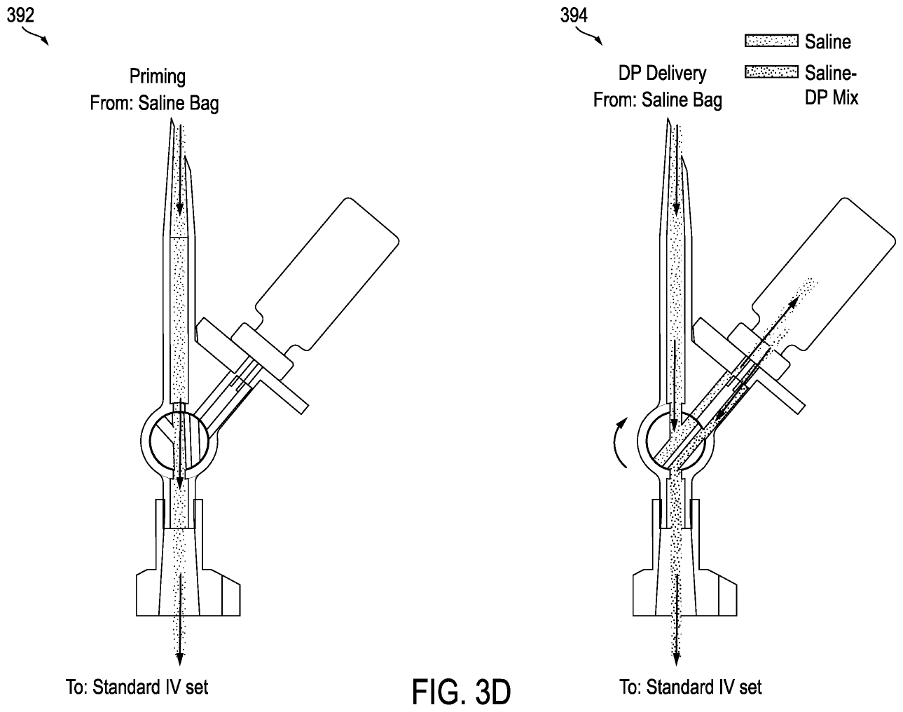

FIG. 3A-FIG. 3C illustrate aspects of a drug product delivery apparatus 300 consistent with implementations of the current subject matter. FIG. 3A is a perspective view and FIG. 3B and FIG. 3C are cross-sectional views of the drug product delivery apparatus 300.

Similar to the drug product delivery apparatus 200, the drug product delivery apparatus 300 includes a central interface member 310 having a cavity 312 surrounded by an outer wall 314 and a plurality of access points 316 formed through respective surfaces of the outer wall 314. Shown in FIG. 3B and FIG. 3C are four access points: a first access point 316a, a second access point 316b, a third access point 316c, and a fourth access point 316d.

The drug product delivery apparatus 300 includes movable conduits 370, as shown in the cross-sectional views of FIG. 3B and FIG. 3C. The movable conduits 370 are positioned within the cavity 312 and interface with the plurality of access points 316 as further described herein.

The drug product delivery apparatus 300 also includes an infusion port 320, a saline port 330, a drug product port 340, and a flush port 345. According to aspects of the current subject matter, each of the ports 320, 330, 340, and 345 has a first end and a second end, and each of the ports 320, 330, 340, and 345 is coupled at one end to a respective access point 316a, 316b, 316c, and 316d of the central interface member 310.

The infusion port 320 has a first end 321, coupled to the first access point 316a of the central interface member 310, and a second end 322. The second end 322 of the infusion port 320 may be configured to connect to an IV administration set for delivering fluid from the infusion port 320.

The saline port 330 has a first end 331 and a second end 332. The second end 332 of the saline port 330 is coupled to the second access point 316b of the central interface member 310.

Consistent with implementations of the current subject matter, the movable conduits 370 are configured to move between at least a first position and a second position. In the first position, the movable conduits 370 interface with the plurality of access points 316 such that a first passageway 350 is defined between the second access point 316b and the first access point 316a to provide fluid connection between the saline port 330 and the infusion port 320.

The first end 331 of the saline port 330 is configured to be coupled to a saline source, thus allowing the saline port 330 to deliver saline from the saline source through the first passageway 350, formed by the movable conduits 370, to the infusion port 320. In some implementations, the saline port 330 is terminated with a spike for coupling to the saline source (e.g., a saline bag or the like).

The drug product port 340 of the drug product delivery apparatus 300 has a first end 341 and a second end 342. The second end 342 of the drug product port 340 is coupled to the third access point 316c of the central interface member 310.

The flush port 345 has a first end 346 and a second end 347. The second end 347 of the flush port 345 is coupled to the fourth access point 316d of the central interface member 310.

Consistent with implementations of the current subject matter, in the second position, the movable conduits 370 interface with the plurality of access points 316 such that a second passageway 360 is defined between the third access point 316c and the first access point 316a to provide fluid connection between the drug product port 340 and the infusion port 320. Moreover, in the second position, the movable conduits 370 interface with the plurality of access points 316 such that a third passageway 365 is defined between the second access point 316b and the fourth access point 316d to provide fluid connection between the saline port 330 and the flush port 345.

The first end 341 of the drug product port 340 is configured to be coupled to a drug product source, thus allowing the drug product port 340 to deliver drug product from the drug product source through the second passageway 360 to the infusion port 320.

The first end 346 of the flush port 345 is configured to be coupled to the drug product source, thus providing for saline to flows from the saline port 330, through the third passageway 365, to the drug product source. The third passageway 365 thereby allows, through the connection of the saline source and the drug product source, for flushing to occur within the drug product source.

As shown in FIG. 3A-FIG. 3C, the first end 341 of the drug product port 340 and the first end 346 of the flush port 345 may terminate at and/or be engaged with, a vial adaptor 380 configured to engage with a vial (not shown in FIG. 3A-FIG. 3C). Consistent with implementations of the current subject matter, saline flows from the saline source, through the vial adaptor 380, to the vial to mix with and flush the drug product contained in the vial. Moreover, the drug product flows from the vial and the vial adaptor 380, through the drug product port 340 and the second passageway 360 to the infusion port 320.

Consistent with implementations of the current subject matter, the drug product delivery apparatus 300 may include a control member 390, as shown in FIG. 3A. The control member 390 is coupled to the movable conduits 370 to move the movable conduits 370 between the first position and the second position. For example, the control member 390 may be configured such that movement, such as rotation, of the control member 390 causes the movable conduits 370 to rotate within the cavity 312 of the central interface member 310. Thus, movement of the control member 390 causes the movable conduits 370 to be aligned to form the first passageway 350, the second passageway 360, and the third passageway 365.

According to aspects of the current subject matter, the first position of the movable conduits 370 and the second position of the movable conduits 370 do not concurrently occur. That is, while the first passageway 350 is formed and open between the second access point 316b and the first access point 316a to provide fluid connection between the saline port 330 and the infusion port 320, the second passageway 360 and the third passageway 365 are not formed (e.g., the movable conduits 370 are positioned in such a way that there is no fluid connection between the drug product port 340 and the infusion port 320, and between the saline port 330 and the flush port 445, and). When the second passageway 360 and the third passageway 365 are formed and open, the first passageway 350 is not formed (e.g., the movable conduits 370 are positioned in such a way that there is no fluid connection between the saline port 330 and the infusion port 320). This arrangement allows for saline priming to occur between the saline port 330 and the infusion port 320, separate from delivery of the drug product from the drug product port 340 to the infusion port 320 and saline flushing into the drug product source via the flush port 345.

FIG. 3D illustrate aspects of operation of the drug product delivery apparatus 300, consistent with implementations of the current subject matter. Drug product delivery utilizing the drug product delivery apparatus 300 includes, according to some implementations, two stages, as shown in FIG. 3D: priming 392 and drug product delivery 394. The drug product delivery apparatus 300 is shown in cross-sectional views to illustrate aspects of the movable conduits 370, the first passageway 350, the second passageway 360, and the third passageway 365. Each of the stages is accomplished via pumping from an infusion pump that interfaces with the IV administration set coupled to the infusion port 320.

In the priming stage 392, the movable conduits 370 are positioned in the first position such that the first passageway 350 is formed between the saline port 330 and the infusion port 320, allowing saline to flow from the saline source (e.g., a saline bag), through the first passageway 350, to the infusion port 320 (coupled to an IV administration set). The priming 392 involves infusing, via the saline port 330, a first quantity of saline into the drug product delivery apparatus 300. The saline port 330 may be terminated with a spike or the like for coupling to the saline source, such as the saline bag. The priming provides for the first quantity of saline to be distributed to the infusion port 320 through the first passageway 350.

For the stage of the drug product delivery 394, the movable conduits 370 are moved into the second position. For example, the control member 390 is rotated such that the second passageway 360 and the third passageway 365 are formed. This allows drug product to flow from the vial to the drug product port 340, through the second passageway 360, to the infusion port 320 (coupled to the IV administration set); and for saline to flow from the saline port 330, through the third passageway 365, and to the flush port 345 and the drug product source.

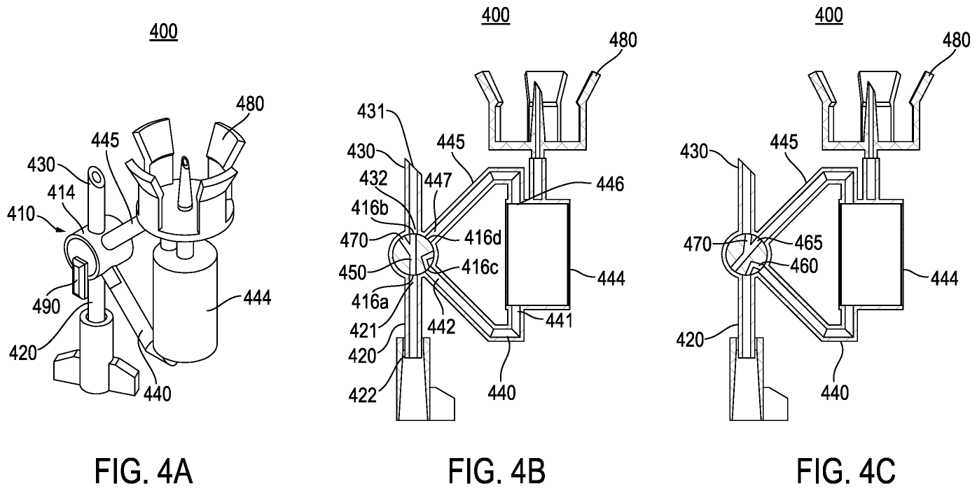

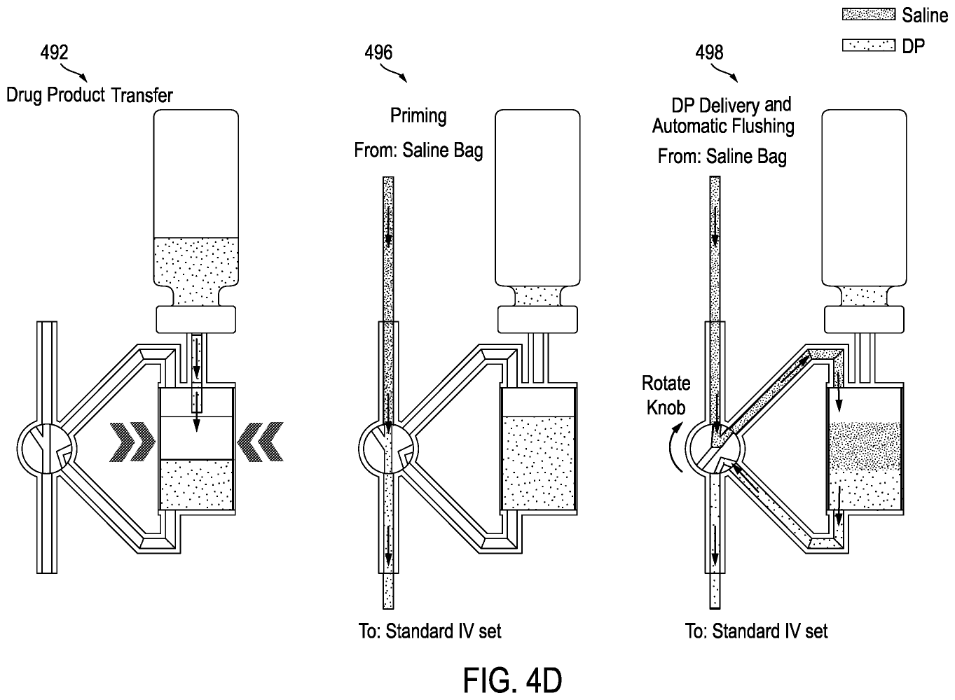

FIG. 4A-FIG. 4C illustrate aspects of a drug product delivery apparatus 400 consistent with implementations of the current subject matter. FIG. 4A is a perspective view and FIG. 4B and FIG. 4C are cross-sectional views of the drug product delivery apparatus 400.

Similar to the drug product delivery apparatus 300, the drug product delivery apparatus 400 includes a central interface member 410 having a cavity 412 surrounded by an outer wall 414 and a plurality of access points 416 formed through respective surfaces of the outer wall 414. Shown in FIG. 4B and FIG. 4C are four access points: a first access point 416a, a second access point 416b, a third access point 416c, and a fourth access point 416d.

The drug product delivery apparatus 400 includes movable conduits 470, as shown in the cross-sectional views of FIG. 4B and FIG. 4C. The movable conduits 470 are positioned within the cavity 412 and interface with the plurality of access points 416 as further described herein.

The drug product delivery apparatus 400 also includes an infusion port 420, a saline port 430, a drug product port 440, and a flush port 445. According to aspects of the current subject matter, each of the ports 420, 430, 440, and 445 has a first end and a second end, and each of the ports 420, 430, 440, and 445 is coupled at one end to a respective access point 416a, 416b, 416c, and 416d of the central interface member 410.

The infusion port 420 has a first end 421, coupled to the first access point 416a of the central interface member 410, and a second end 422. The second end 422 of the infusion port 420 may be configured to connect to an IV administration set for delivering fluid from the infusion port 420.

The saline port 430 has a first end 431 and a second end 432. The second end 432 of the saline port 430 is coupled to the second access point 416b of the central interface member 410.

Consistent with implementations of the current subject matter, the movable conduits 470 are configured to move between at least a first position and a second position. In the first position, the movable conduits 470 interface with the plurality of access points 416 such that a first passageway 450 is defined between the second access point 416b and the first access point 416a to provide fluid connection between the saline port 430 and the infusion port 420.

The first end 431 of the saline port 430 is configured to be coupled to a saline source, thus allowing the saline port 430 to deliver saline from the saline source through the first passageway 450, formed by the movable conduits 470, to the infusion port 420. In some implementations, the saline port 430 is terminated with a spike for coupling to the saline source (e.g., a saline bag or the like).

The drug product port 440 of the drug product delivery apparatus 400 has a first end 441 and a second end 442. The second end 442 of the drug product port 440 is coupled to the third access point 416c of the central interface member 410.

The flush port 445 has a first end 446 and a second end 447. The second end 447 of the flush port 445 is coupled to the fourth access point 416d of the central interface member 410.

Consistent with implementations of the current subject matter, in the second position, the movable conduits 470 interface with the plurality of access points 416 such that a second passageway 460 is defined between the third access point 416c and the first access point 416a to provide fluid connection between the drug product port 440 and the infusion port 420. Moreover, in the second position, the movable conduits 470 interface with the plurality of access points 416 such that a third passageway 465 is defined between the second access point 416b and the fourth access point 416d to provide fluid connection between the saline port 430 and the flush port 445.

The first end 441 of the drug product port 440 is configured to be coupled to a drug product source 444, thus allowing the drug product port 440 to deliver drug product from the drug product source 444 through the second passageway 450 to the infusion port 420. According to some implementations of the current subject matter, the drug product source 444 is a chamber in which an infusion volume of a drug product is contained. For example, the drug product source 444 may connect to a vial or a container through, for example, a vial adaptor 480 configured to engage with the vial or the container (not shown in FIG. 4A-FIG. 4C). In some implementations in which the drug product source 444 is a chamber, the chamber may be a squeezable or flexible chamber that allows for transferring the drug product from the vial or the container to the drug product source via the vial adaptor 480. The chamber may be a syringe, a flexible chamber, or a chamber with an extending member configured to withdraw an infusion volume of the drug product from the vial or the container. In some implementations, the vial adaptor 480 is not required.

The first end 446 of the flush port 445 is configured to be coupled to the drug product source 444, thus providing for saline to flows from the saline port 430, through the third passageway 465, to the drug product source 444. The third passageway 465 thereby allows, through the connection of the saline source and the drug product source 444, for flushing to occur within the drug product source 444.

Consistent with implementations of the current subject matter, the first end 446 of the flush port 445 may be coupled to an upper portion of the drug product source 444. The upper portion may refer to a portion of the drug product source 444 above a mid-point along a length of the drug product source 444. The upper portion may refer to a top surface of the drug product source 444. The first end 441 of the drug product port 440 may be coupled to a lower portion of the drug product source 444. The lower portion may refer to a portion of the drug product source 444 below a mid-point along a length of the drug product source 444. The lower portion may refer to a bottom surface of the drug product source 444. The configuration in which the flush port 445 is coupled to an upper portion of the drug product source 444 and the drug product port 440 is coupled to a lower portion provides for saline to be added to the drug product source 444 for automatic flushing. The drug product flows from the drug product source 444, through the drug product port 440 and the second passageway 460 to the infusion port 420.

Consistent with implementations of the current subject matter, the drug product delivery apparatus 400 may include a control member 490, as shown in FIG. 4A. The control member 490 is coupled to the movable conduits 470 to move the movable conduits 470 between the first position and the second position. For example, the control member 490 may be configured such that movement, such as rotation, of the control member 490 causes the movable conduits 470 to rotate within the cavity 412 of the central interface member 410. Thus, movement of the control member 490 causes the movable conduits 470 to be aligned to form the first passageway 450, the second passageway 460, and the third passageway 465.

According to aspects of the current subject matter, the first position of the movable conduits 470 and the second position of the movable conduits 470 do not concurrently occur. That is, while the first passageway 450 is formed and open between the second access point 416b and the first access point 416a to provide fluid connection between the saline port 430 and the infusion port 420, the second passageway 460 and the third passageway 465 are not formed (e.g., the movable conduits 470 are positioned in such a way that there is no fluid connection between the drug product port 440 and the infusion port 420, and between the saline port 430 and the flush port 445). When the second passageway 460 and the third passageway 465 are formed and open, the first passageway 450 is not formed (e.g., the movable conduits 470 are positioned in such a way that there is no fluid connection between the saline port 430 and the infusion port 420). This arrangement allows for saline priming to occur between the saline port 430 and the infusion port 420, separate from delivery of the drug product from the drug product port 440 to the infusion port 420 and saline flushing into the drug product source 444 via the flush port 445.

FIG. 4D illustrate aspects of operation of the drug product delivery apparatus 400, consistent with implementations of the current subject matter. Drug product delivery utilizing the drug product delivery apparatus 400 includes, according to some implementations, three stages, as shown in FIG. 4D: drug product transfer 492, priming 494, and drug product delivery and flushing 496. The drug product delivery apparatus 400 is shown in cross-sectional views to illustrate aspects of the movable conduits 470, the first passageway 450, the second passageway 460, and the third passageway 465. Each of the stages is accomplished via pumping from an infusion pump that interfaces with the IV administration set coupled to the infusion port 420.

In the drug product transfer stage 492, the drug product is transferred from a vial to the drug product source 444. Consistent with implementations of the current subject matter, the drug product source 444 may be a flexible chamber that when squeezed causes air from the chamber to be forced into the vial, forming a vacuum in the chamber. When the chamber is released, the drug product is transferred into the chamber. A dose line or the like may be provided on the drug product source 444, ensuring dose accuracy by providing a mechanism for transferring a proper amount.

In the priming stage 494, the movable conduits 470 are positioned in the first position such that the first passageway 450 is formed between the saline port 430 and the infusion port 420, allowing saline to flow from the saline source (e.g., a saline bag), through the first passageway 450, to the infusion port 420 (coupled to an IV administration set). The priming 492 involves infusing, via the saline port 430, a first quantity of saline into the drug product delivery apparatus 400. The saline port 430 may be terminated with a spike or the like for coupling to the saline source, such as the saline bag. The priming provides for the first quantity of saline to be distributed to the infusion port 420 through the first passageway 450.

For the drug product delivery and flushing stage 496, the movable conduits 470 are moved into the second position. For example, the control member 490 is rotated such that the second passageway 460 and the third passageway 465 are formed. This allows drug product to flow from the drug product source 444 to the drug product port 440, through the second passageway 460, to the infusion port 420 (coupled to the IV administration set); and for saline to flow from the saline port 430, through the third passageway 465, and to the flush port 445 and the drug product source 444.

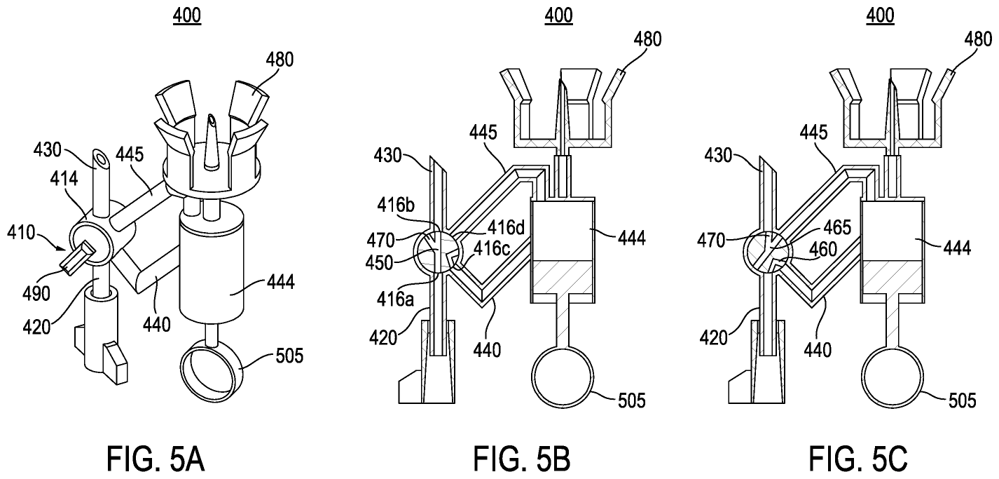

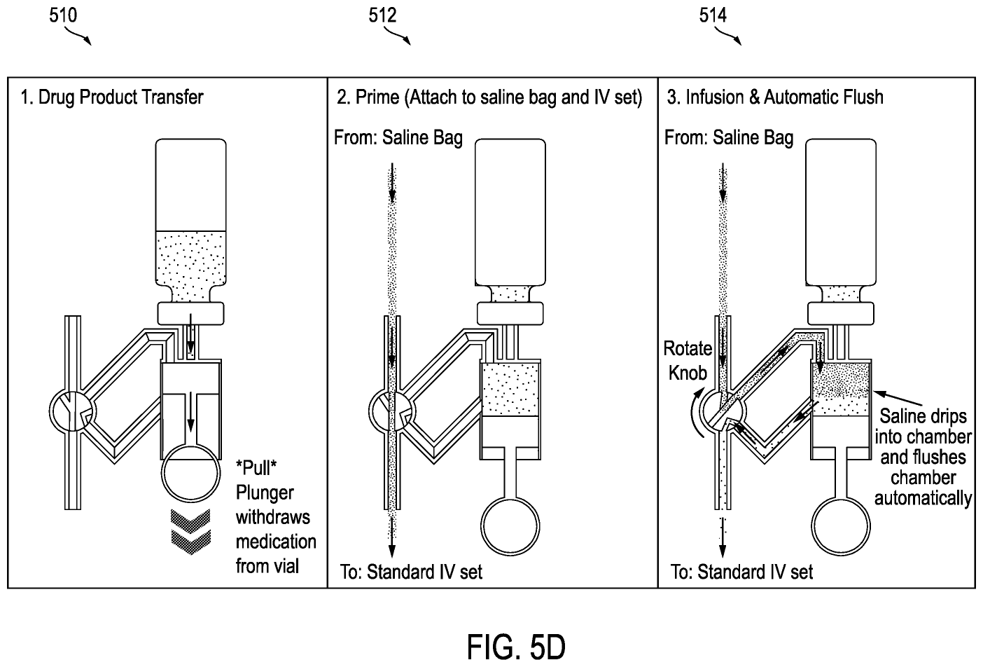

FIG. 5A-FIG. 5C illustrate additional aspects of the drug product delivery apparatus 400 consistent with implementations of the current subject matter. FIG. 5A is a perspective view and FIG. 5B and FIG. 5C are cross-sectional views of the drug product delivery apparatus 400.

In some implementations of the current subject matter, the drug product delivery apparatus 400 may include an extending member 505. The extending member 505 may be, for example, a plunger or the like coupled to the drug product source 444. For example, the extending member 505 may include a base with an arm extending therefrom. The extending member 505 may fit within the drug product source 444, with the base securely but movably contained within. For example, the diameter and circumference of the base may be slightly less than that of the inner area of the drug product source 444, allowing for the base to fit within the drug product source 444 and be moved along a length with a proper amount of force applied. The arm may extend through an opening of a bottom end of the drug product source 444. The base may be moved within the drug product source 444 by moving the arm. For example, the arm may be extended downward to move the base downward, and the arm may be pushed upward to push the base upward.

As the drug product source 444 is connected to a vial or container through, for example, the vial adaptor 480 configured to engage with the vial or the container (not shown in FIG. 5A-FIG. 5C), movement of the extending member 505 may provide for transferring drug product from the vial or container to the drug product source 444. The downward movement of the extending member may cause the drug product to be withdrawn from the vial or the container.

FIG. 5D illustrate aspects of operation of the drug product delivery apparatus 400 including the extending member 505, consistent with implementations of the current subject matter. Drug product delivery utilizing the drug product delivery apparatus 400 includes, according to some implementations, three stages, as shown in FIG. 5D: drug product transfer 510, priming 512, and drug product delivery and flushing 514. The drug product delivery apparatus 400 is shown in cross-sectional views to illustrate aspects of the movable conduits 470, the first passageway 450, the second passageway 460, and the third passageway 465. Each of the stages is accomplished via pumping from an infusion pump that interfaces with the IV administration set coupled to the infusion port 420.

In the drug product transfer stage 510, the drug product is transferred from a vial to the drug product source 444 through movement of the extending member 505. The extending member 505 is initially in a position in which the base is positioned in an upper portion of the drug product source 444. To transfer the drug product from the vial, the extending member 505 is pulled downward through the drug product source 444 to withdraw the drug product from the vial to the drug product source 444. A dose line or the like may be provided on the drug product source 444, ensuring dose accuracy by providing a mechanism for indicating a proper amount to be transferred.

In the priming stage 512, the movable conduits 470 are positioned in the first position such that the first passageway 450 is formed between the saline port 430 and the infusion port 420, allowing saline to flow from the saline source (e.g., a saline bag), through the first passageway 450, to the infusion port 420 (coupled to an IV administration set). The priming 492 involves infusing, via the saline port 430, a first quantity of saline into the drug product delivery apparatus 400. The saline port 430 may be terminated with a spike or the like for coupling to the saline source, such as the saline bag. The priming provides for the first quantity of saline to be distributed to the infusion port 420 through the first passageway 450.

For the drug product delivery and flushing stage 514, the movable conduits 470 are moved into the second position. For example, the control member 490 is rotated such that the second passageway 460 and the third passageway 465 are formed. This allows drug product to flow from the drug product source 444 to the drug product port 440, through the second passageway 460, to the infusion port 420 (coupled to the IV administration set); and for saline to flow from the saline port 430, through the third passageway 465, and to the flush port 445 and the drug product source 444.

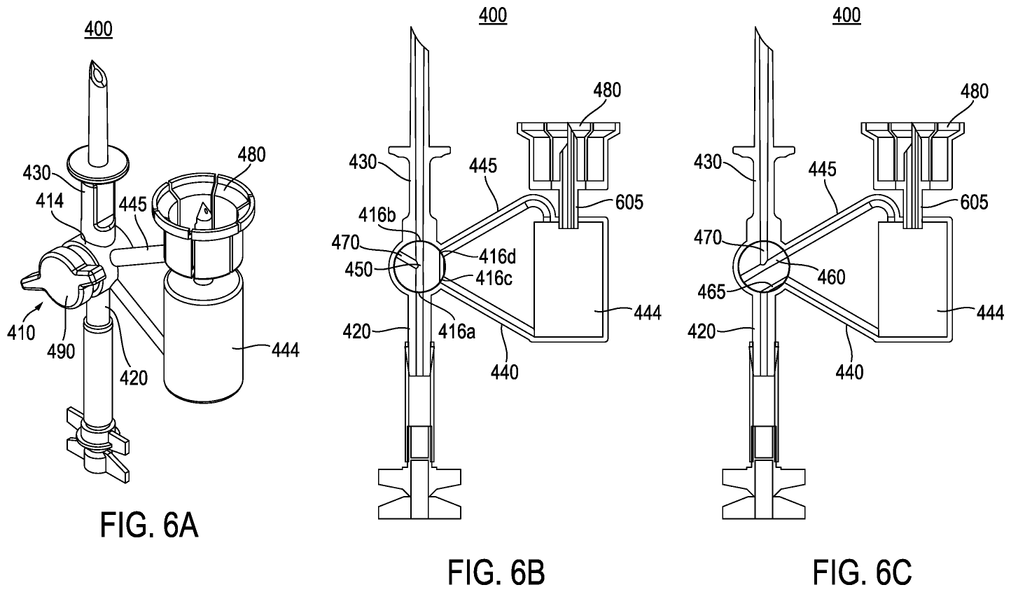

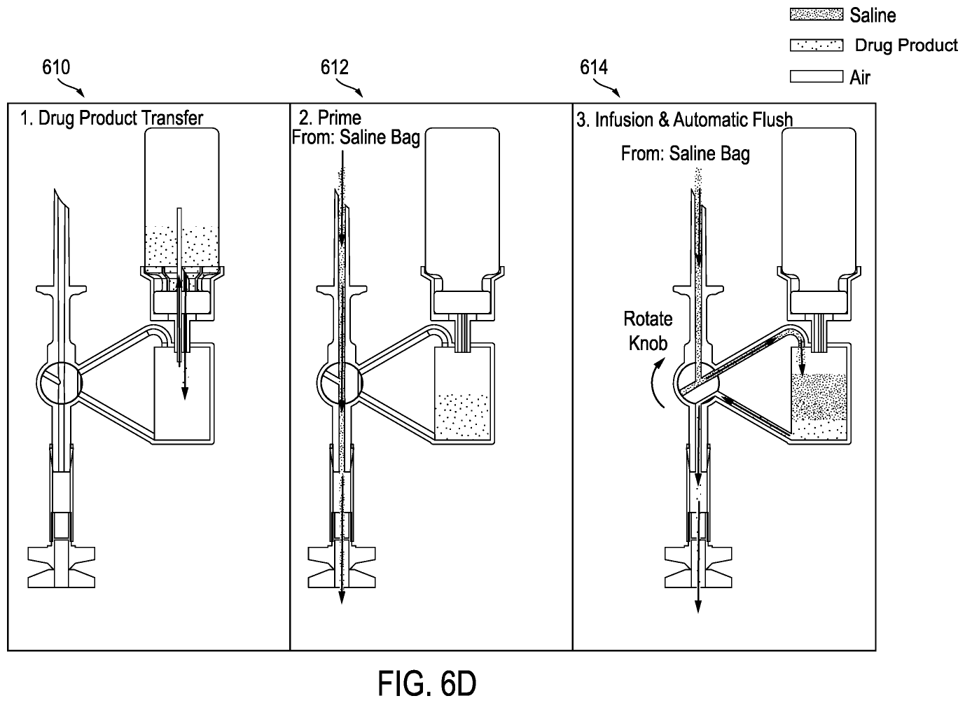

FIG. 6A-FIG. 6C illustrate additional aspects of the drug product delivery apparatus 400 consistent with implementations of the current subject matter. FIG. 6A is a perspective view and FIG. 6B and FIG. 6C are cross-sectional views of the drug product delivery apparatus 400.

In some implementations of the current subject matter, the drug product delivery apparatus 400 may include a dual lumen spike 605 that interfaces between the drug product source 444 and the vial or container engaged with the vial adaptor 480. The dual lumen spike 605 includes two lumens, or pathways, that provide for automatic transfer of the drug product from the vial to the drug product source 444. When the vial is coupled to or inserted in the vial adaptor 480, air flows up one lumen from the drug product source 444 or from a surrounding atmosphere, while the drug product flows down the second lumen to the drug product source 444.

FIG. 6D illustrate aspects of operation of the drug product delivery apparatus 400 including the dual lumen spike 605, consistent with implementations of the current subject matter. Drug product delivery utilizing the drug product delivery apparatus 400 includes, according to some implementations, three stages, as shown in FIG. 6D: drug product transfer 610, priming 612, and drug product delivery and flushing 614. The drug product delivery apparatus 400 is shown in cross-sectional views to illustrate aspects of the movable conduits 470, the first passageway 450, the second passageway 460, and the third passageway 465. Each of the stages is accomplished via pumping from an infusion pump that interfaces with the IV administration set coupled to the infusion port 420.

In the drug product transfer stage 610, the drug product is transferred from a vial to the drug product source 444 through the dual lumen spike 605. When the vial is coupled to or inserted in the vial adaptor 480, air flows up one lumen from the drug product source 444, while the drug product flows down the second lumen to the drug product source 444.

In the priming stage 612, the movable conduits 470 are positioned in the first position such that the first passageway 450 is formed between the saline port 430 and the infusion port 420, allowing saline to flow from the saline source (e.g., a saline bag), through the first passageway 450, to the infusion port 420 (coupled to an IV administration set). The priming 492 involves infusing, via the saline port 430, a first quantity of saline into the drug product delivery apparatus 400. The saline port 430 may be terminated with a spike or the like for coupling to the saline source, such as the saline bag. The priming provides for the first quantity of saline to be distributed to the infusion port 420 through the first passageway 450.

For the drug product delivery and flushing stage 614, the movable conduits 470 are moved into the second position. For example, the control member 490 is rotated such that the second passageway 460 and the third passageway 465 are formed. This allows drug product to flow from the drug product source 444 to the drug product port 440, through the second passageway 460, to the infusion port 420 (coupled to the IV administration set); and for saline to flow from the saline port 430, through the third passageway 465, and to the flush port 445 and the drug product source 444.

In some implementations, the central interface member, the infusion port, the saline port, the drug product port, and the flush port (when incorporated) are a single molded component formed of one or more resilient materials. In some implementations, one or more of the infusion port, the saline port, the drug product port, and the flush port are individual components configured to securely mate with respective ones of the access points. For example, one or more of the infusion port, the saline port, the drug product port, and the flush port may snap fit or screw into or otherwise connect to respective ones of the access points. In some implementations, the central interface member may be an expandable and/or squeezable chamber. In some implementations, the central interface member may be integral with a saline source, a drug product source, and/or an IV administration set.

The drug product source may, according to some aspects of the current subject matter, include a plurality of vials. Consistent with implementations of the current subject matter, a plurality of vial adaptors, each configured to engage with a respective one of the plurality of vials, may be connected in series. For example, a first vial adaptor may connect with or be positioned at the first end of the drug product port. A second vial adaptor may connect with the first vial adaptor. The first vial adaptor and the second vial adaptor may be connected such that drug product from a first vial, engaged with the first vial adaptor, and a second vial, engaged with the second vial adaptor, flow through the drug product port into the cavity of the central interface member.

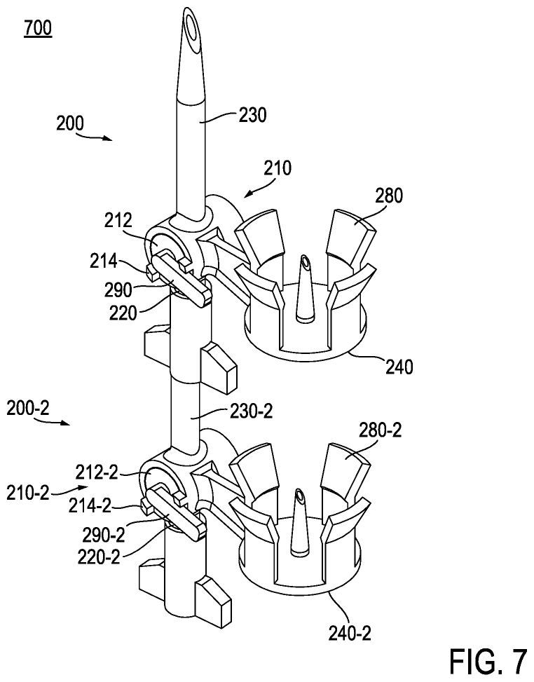

With reference to FIG. 7, aspects of a drug product delivery apparatus 700 are shown. The drug product delivery apparatus 700 includes two separate ones of the drug product delivery apparatus 200 (a first drug product delivery apparatus 200 and a second drug product delivery apparatus 200-2) coupled to one another to provide for delivery of two drug products. A second central interface member 210-2 has a second cavity 212-2 surrounded by a second outer wall 214-2, and a plurality of second access points (not shown in FIG. 7) formed through respective surfaces of the second outer wall 214-2.

A second infusion port 220-2 has a first end and a second end, the first end coupled to a first access point of the plurality of second access points. The second end of the second infusion port 220-2 is coupled to an IV administration set (not shown) for delivery of fluids to a patient.

A second saline port 230-2 has a first end and a second end. The second end of the second saline port 230-2 is coupled to a second access point of the plurality of second access points. The first end of the second saline port 230-2 is coupled to the second end of the infusion port 220 to fluidly connect the infusion port 220 of the first drug product delivery apparatus 200 and the second saline port 230-2 of the second drug product delivery apparatus 200-2.

A second drug product port 240-2 has a first end and a second end. The second end of the second drug product port 240-2 is coupled to a third access point of the plurality of second access points to fluidly connect the second drug product port 240-2 and the second infusion port 220-2. The first end of the second drug product port 240-2 may be, or may be engaged with, a second vial adaptor 280-2 configured to engage with a second vial (not shown in FIG. 7) such that a second drug product flows from the second vial and the second vial adaptor 280-2, through the second drug product port 240-2 to the second infusion port 220-2 to deliver the second drug product.

Consistent with implementations of the current subject matter, the second drug product delivery apparatus 200-2 may include a control member 290-2, as shown in FIG. 7. The second control member 290-2 is coupled to movable conduits within the second cavity 212-2 to form passageways between the second ports (220-2, 230-2, and 240-2) in a manner equivalent to that described with reference to the drug product delivery apparatus 200. Consistent with implementations of the current subject matter, by controlling movement of the control member 290 and the second control member 290-2, priming, drug delivery of the drug product and the second drug product, and flushing states may be implemented. For example, the drug product in a vial coupled to the drug delivery apparatus 200 may be delivered, followed by the second drug product in the second vial coupled to the second drug product delivery apparatus 200-2; or the reverse order may be implemented. In some implementations, the drug product and the second drug product may be delivered at the same time. In some implementation, a flushing step may be incorporated between delivery of the drug product and the second drug product, or between delivery of the second drug product and the drug product.

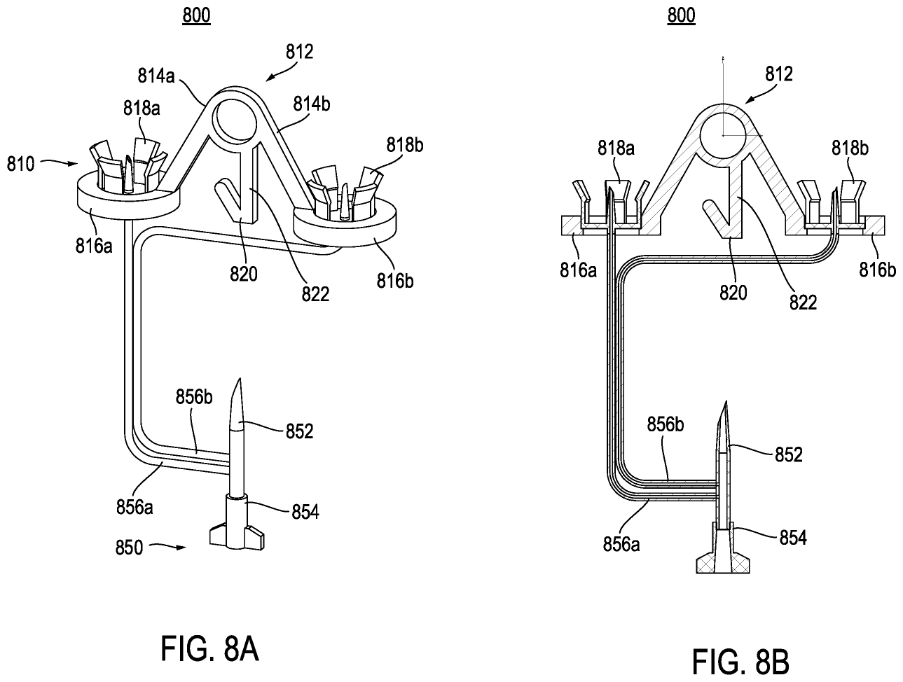

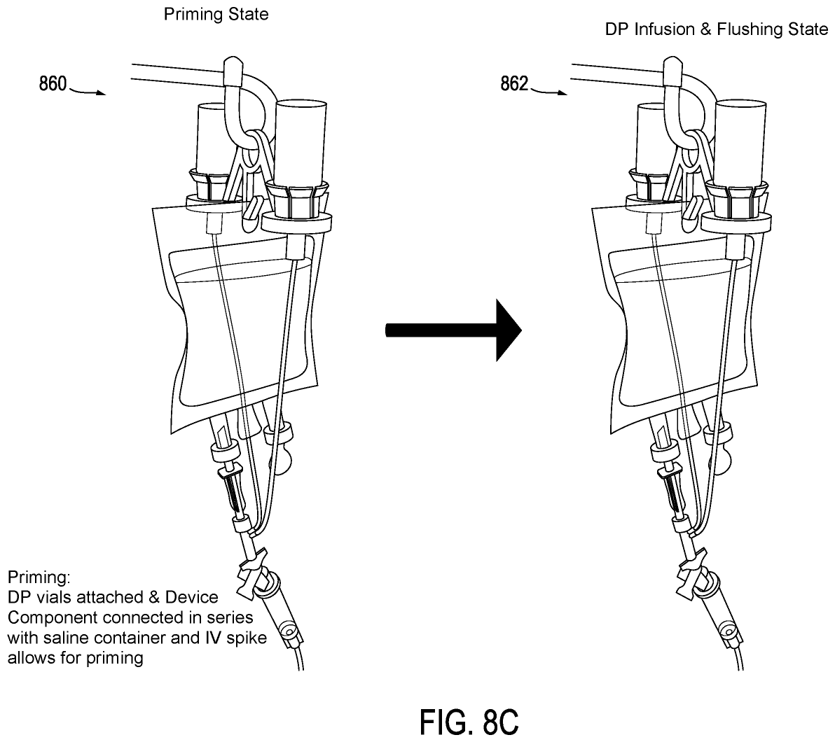

FIG. 8A-FIG. 8B are diagrams illustrating aspects of a drug product delivery apparatus 800 consistent with additional implementations of the current subject matter. FIG. 8A is a perspective view of the drug product delivery apparatus 800, and FIG. 8B is a cross-sectional view.

According to implementations of the current subject matter, the drug product delivery apparatus 800 includes a connection component 810 and a port manifold 850. The connection component 810 serves to support, in an upright configuration, a saline source containing saline and one or more vials containing one or more drug products to be infused into a patient. The port manifold 850 provides a connection between the saline source and the one or more vials supported by the connection component 810 and an IV administration set, as further described herein.

The connection component 810 includes a central connection member 812 configured to connect to an infusion stand or the like. The central connection member 812 may be, for example, a hooked or ringed shape. The central connection member 812 may include an attachment structure with at least a partial opening from which one or more support arms extend, where the attachment structure is oriented such that the at least partial opening is generally aligned with a vertical axis.

The connection component 810 further includes a first vial connection member 816a coupled to a first support arm 814a. According to some aspects of the current subject matter, the first support arm 814a extends from the central connection member 812. The first vial connection member 816a is configured to support a first vial adaptor 818a for engaging with a first vial. In some implementations, the first vial connection member 816a may directly or indirectly support the first vial without use of the vial adaptor. In some implementations, the first vial adaptor 818a may be integrated within the first vial connection member 816a. The first vial connection member 816a may include a ringed structure into which the first vial adaptor 818a fits or is adapted, where the ringed structure is oriented such that an opening thereof is generally aligned with a horizontal axis.

Shown in FIG. 8A and FIG. 8B are a second vial connection member 816b, a second support arm 814b, and a second vial adaptor 818b. However, implementations of the current subject matter are not limited to two sets of support arms/vial connection members/vial adaptors. Rather, in some implementations, one support arm, vial connection member, and vial adaptor may be incorporated, and in some implementations two or more may be incorporated. The central connection member 812 and the support arms may be adapted to accommodate any number of sets of support arms/vial connection members/vial adaptors. For example, for three sets, the support arms may be spaced apart by 120 degrees around the central connection member 812. For a single set, the support arm may extend longitudinally downward in a vertical manner from the central connection member. Possible other adaptations are within the scope of the current implementations of the drug product delivery apparatus 800.

The connection component 810 further includes a saline source connection member 820 coupled to a support arm 822. The support arm 822 extends from the central connection member 812 and is configured to support a saline source, such as a saline bag or the like. For example, a distal end portion of the saline source connection member 820 may be of a hooked or similar shape on which the saline source may be securely retained during an infusion.

Consistent with implementations of the current subject matter, when the first vial is connected to and/or engaged with the first vial adaptor 818a (and a second vial is connected to and/or engaged with the second vial adaptor 818b if included) and when the saline source is coupled to the saline source connection member 820, the configuration of the connection component 810 is such that the first vial (and the second vial if included) are positioned above the saline source when referring to a vertical axis. That is, the first vial (and the second vial if included) are higher than the saline source. This arrangement facilitates infusion of the drug products contained in the first vial (and the second vial if included).

As noted, the drug product delivery apparatus 800 also includes the port manifold 850 to connect the saline source and the one or more vials with the IV administration set. The port manifold 850 includes a port 852 configured to be inserted into the saline source (connected to the saline source connection member 820). The port 852 may terminate with a spike for coupling with the saline source. An infusion line 854 is configured to provide a passageway between the port 852 and an intravenous administration set. Additionally, a first drug product line 856a is provided. The first drug product line 856a is configured to connect to the first vial adaptor 818a at a first end and to the infusion line 854 at the second end. In implementations with a second vial adaptor 818b, a second drug product line 856b is provided, and is configured to connect to the second vial adaptor 818b at a first end and to the infusion line 854 at the second end.

Consistent with implementations of the current subject matter, the infusion line 854 may be closed from the first drug product line 856a via a valve positioned at an interface between the first drug product line 856a and the infusion line 854. The valve may be movable by a rotating member between a first position, in which the infusion line 854 is closed from the first drug product line 856a, and a second position, in which the infusion line 854 is open to the first drug product line 856a. The valve, or a separate valve, may also interface with the second drug product line 856b at a junction with the infusion line 854, where the valve functions to close and open the infusion line 854 with respect to the second drug product line 856b.

The port manifold 850 may further include a removable attachment member at an end of the infusion line 854. Removal of the removable attachment member may provide for attachment of the infusion line 854 to the IV administration set.

FIG. 8C illustrate aspects of operation of the drug product delivery apparatus 800, consistent with implementations of the current subject matter. Drug product delivery utilizing the drug product delivery apparatus 800 includes, according to some implementations, two stages, as shown in FIG. 8C: priming 860 and drug product delivery and flushing 862. Each of the stages is accomplished via pumping, e.g. from an infusion pump that interfaces with the IV administration set coupled to the infusion line. The drug product delivery apparatus 800, as shown in FIG. 8C, is prepared with two vials, a first vial and a second vial, engaged in respective ones of the first vial adaptor 818a and the second vial adaptor 818b, and a saline bag supported by the saline source connection member 820.

The priming stage 860 involves infusing the infusion line 854 with a first quantity of saline from the saline source. The drug product delivery and flushing stage 862 includes opening an interface (via, for example, the valve) between the first drug product line 856a and/or the second drug product line 856b and the infusion line 854, allowing for flow of the first drug product and/or the second drug product through the first drug product line 856a and/or the second drug product line 856b and the infusion line 854 to the IV administration set. As the first vial and/or the second vial are positioned at a higher vertical position than the saline source, the first drug product and/or the second drug product will flow, until emptied, through the infusion line 854, at which point the saline in the saline source will flush the infusion line 854.

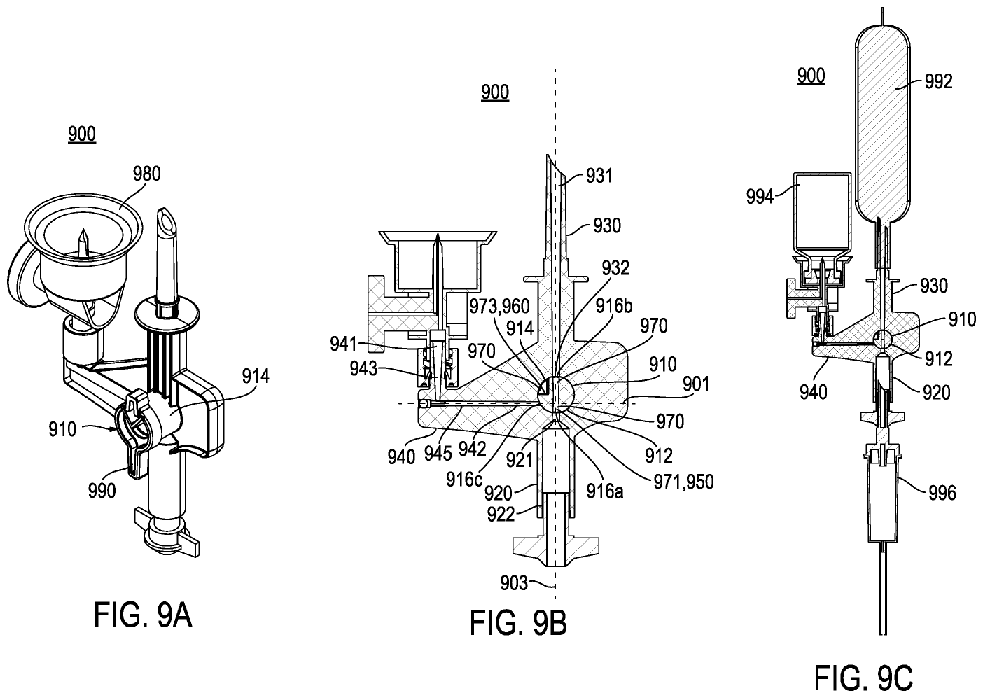

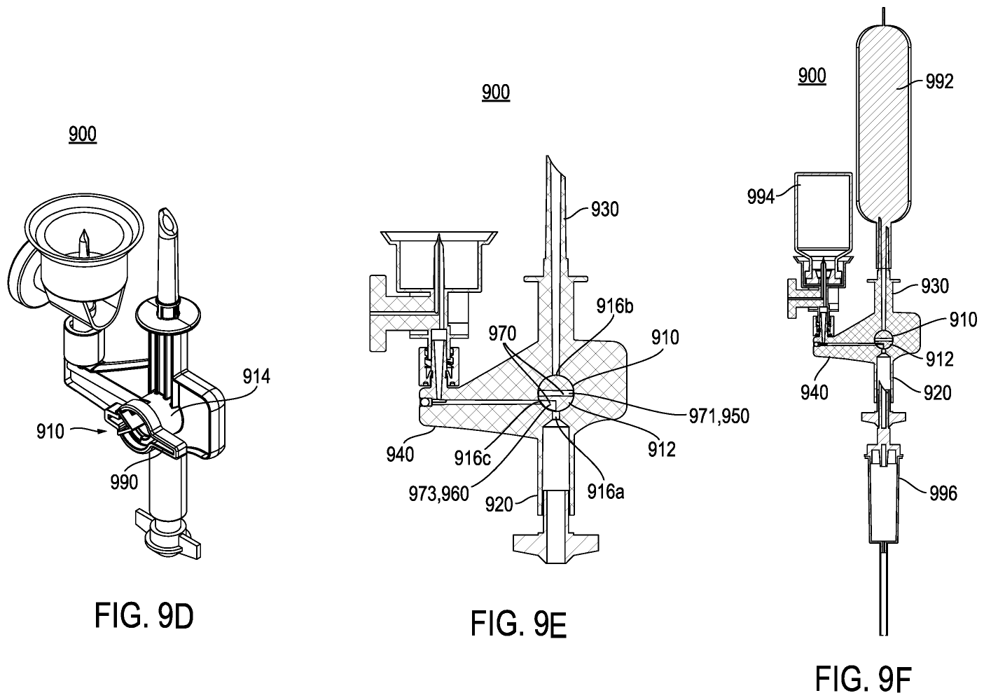

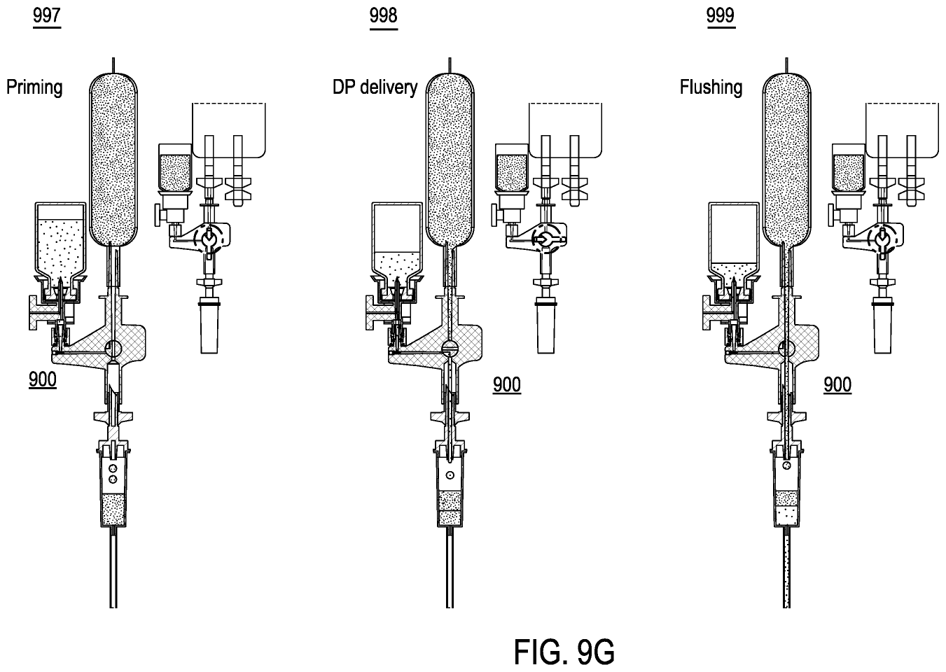

FIG. 9A-FIG. 9G illustrate aspects of a drug product delivery apparatus 900 consistent with implementations of the current subject matter. FIG. 9A is a perspective view of the drug delivery apparatus 900 in a first position and FIG. 9B and FIG. 9C are cross-sectional views of the drug product delivery apparatus 900 in the first position. FIG. 9D is a perspective view of the drug delivery apparatus 900 in a second position and FIG. 9E and FIG. 9F are cross-sectional views of the drug product delivery apparatus 900 in the second position. FIG. 9C and FIG. 9F show the drug product delivery apparatus 900 coupled to a saline source 992 and a vial 994 containing a drug product.

The drug product delivery apparatus 900 includes a central interface member 910. The central interface member 910 includes a cavity 912 surrounded by an outer wall 914 and a plurality of access points formed through respective surfaces of the outer wall 914. For example, the plurality of access points may include one, two, three, four, or more access points. Referring to FIGS. 9B, 9C, 9E, and 9F, the central interface member 910 may include at least three access points, such as a first access point 916a, a second access point 916b, and a third access point 916c. The access points may provide access to the cavity 912, and may allow for a fluid to enter and/or exit the cavity 912 of the central interface member 910.

The drug product delivery apparatus 900 also includes an infusion port 920, a saline port 930, and a drug product port 940. According to aspects of the current subject matter, each of the ports 920, 930, and 940 has a first end and a second end, and each of the ports 920, 930, and 940 is coupled at one end to a respective access point 916a, 916b, and 916c of the central interface member 910.

The infusion port 920 has a first end 921, coupled to the first access point 916a of the central interface member 910, and a second end 922. The second end 922 may be opposite the first end 921. The second end 922 of the infusion port 920 may connect to an IV administration set 996 for delivering fluid from the infusion port 920. For example, the fluid passing through the cavity 912 of the central interface member 910 may be delivered to the IV administration set 996 via the infusion port 920. The infusion port 920 may be coupled to a luer lock or other connector, which is coupled to tubing or another delivery mechanism for delivering the fluid to the patient.

The saline port 930 has a first end 931 and a second end 932. The second end 932 may be opposite the first end 931. The second end 932 of the saline port 930 is coupled to the second access point 916b of the central interface member 910. The first end 931 of the saline port 930 is configured to be coupled to a saline source. Thus, saline from the saline source may pass through the saline port 930, into and through the cavity 912, and through the infusion port 920 to be delivered to the IV administration set 996 to prime and/or flush the IV administration set 996. In some implementations, the saline port 930 is terminated with a spike for coupling to the saline source (e.g., a saline bag or the like).

The saline port 930 may be vertically aligned with the infusion port 920. For example, the saline port 930 and the infusion port 920 may be aligned along a central longitudinal axis 903 of the central interface member 910. In some implementations, the saline port 930 and the infusion port 920 are positioned directly opposite from one another across the central interface member 910. Such configurations allow for the saline to easily flow between the saline port 930 and the infusion port 920.

The drug product port 940 of the drug product delivery apparatus 900 has a first end 941 and a second end 942. The second end 942 of the drug product port 940 is coupled to the third access point 916c of the central interface member 910. In some implementations, the second end 942 and the first end 941 are positioned along a single axis.

In some implementations, such as in the configuration shown in FIGS. 9A-9F, the first end 941 is positioned in a direction that is perpendicular relative to the second end 942. For example, the drug product port 940 may include a first drug product passageway 943 and a second drug product passageway 945. The first drug product passageway 943 and the second drug product passageway 945 may be fluidly coupled and/or integrally formed to define the drug product port 940. The first drug product passageway 943 may extend between the first end 941 and the second drug passageway 945. The second drug product passageway 945 may extend between the first drug product passageway 943 and the second end 942. The second drug product passageway 945 may extend laterally from the central interface member 910, such as along a lateral axis 901 of the central interface member 910 that is perpendicular to the longitudinal axis 903 of the central interface member 910. In some implementations, such as in the example drug product deliver apparatus 200 shown in FIG. 2B and FIG. 2C, the second drug product passageway 945 may extend at an angle relative to the longitudinal axis 903 of the central interface member 910 and/or to the first drug product passageway 943. The first drug product passageway 943 may extend in a direction that is perpendicular relative to the lateral axis 901 and/or to the second drug product passageway 945, though other angles are contemplated consistent with implementations of the current subject matter.

Referring again to FIGS. 9A-9F, the first end 941 of the drug product port 940 is configured to be coupled to a drug product source, thus allowing the drug product port 940 to deliver drug product from the drug product source through the second passageway 960 to the infusion port 920. The first end 941 of the drug product port 940 may be, or may be engaged with, a vial adaptor 980 configured to engage with the vial 994 (shown in FIG. 9C and FIG. 9F) such that the drug product flows from the vial and the vial adaptor 980, through the drug product port 940, into the cavity 912 of the central interface member 910, and to the infusion port 920.

The drug product delivery apparatus 900 may include movable conduits 970, as shown in the cross-sectional views of FIG. 9B, FIG. 9C, FIG. 9E, and FIG. 9F. The movable conduits 970 are positioned within the cavity 912 and interface with the plurality of access points, such as the first, second, and third access points 916a, 916b, 916c. The movable conduits 970 may include one, two, three, four, five, or more movable conduits 970. For example, as shown in FIGS. 9B, 9C, 9E, and 9F, the movable conduits 970 include a first movable conduit 971 and a second movable conduit 973. Each of the movable conduits 970 may be separated from one another such that each of the movable conduits are not in fluid communication with one another. In other implementations, one or more of the movable conduits 970 may be fluidly coupled with one another.

Consistent with implementations of the current subject matter, the movable conduits 970 are configured to move between at least a first position (see FIGS. 9A-9C) and a second position (see FIGS. 9D-9F). In the first position, saline is permitted to pass through the central interface member 910, such as from the saline source 992 to the IV administration set 996. In the first position, the drug product may not be permitted to pass through the central interface member 910. For example, in the first position, openings providing access to the second movable conduit 973 may not be fluidly connected with the first, second, and/or third access point 916a, 916b, 916c. Instead, the openings into the second movable conduit 973 may be blocked by the outer wall 914 surrounding the cavity 912.

In the second position, the drug product is permitted to pass through the central interface member 910, such as from the vial 994 to the IV administration set 996. In the second position, the saline may not be permitted to pass through the central interface member 910. For example, in the second position, openings providing access to the first movable conduit 973 may not be fluidly connected with the first, second, and/or third access point 916a, 916b, 916c. Instead, the openings into the first movable conduit 971 may be blocked by the outer wall 914 surrounding the cavity 912.

In other words, the first position of the movable conduits and the second position of the movable conduits 970 do not concurrently occur. That is, while the first passageway 950 is formed and open between the second access point 916b and the first access point 916a to provide fluid connection between the saline port 930 and the infusion port 920, the second passageway 960 is not formed (e.g., the movable conduits 970 are positioned in such a way that there is no fluid connection between the drug product port 940 and the infusion port 920). When the second passageway 960 is formed and open between the third access point 916c and the first access point 916a to provide fluid connection between the drug product port 940 and the infusion port 920, the first passageway 950 is not formed (e.g., the movable conduits 970 are positioned in such a way that there is no fluid connection between the saline port 930 and the infusion port 920). This arrangement allows for saline priming and flushing to occur between the saline port 930 and the infusion port 920 separate from delivery of the drug product from the drug product port 940 to the infusion port 920.

Consistent with implementations of the current subject matter, in the first position, the movable conduits 970 interface with the plurality of access points such that a first passageway 950 is defined between the second access point 916b and the first access point 916a to provide fluid connection between the saline port 930 and the infusion port 920. In this configuration, the first movable conduit 971 defines the first passageway 950. The first movable conduit 971 may form a straight (e.g., unbent) channel that extends between the first access point 916a and the second access point 916b. As noted above, the first end 931 of the saline port 930 is configured to be coupled to a saline source, thus allowing the saline port 930 to deliver saline from the saline source through the first passageway 950 formed by the first movable conduit 971 to the infusion port 920.

In the second position, the movable conduits 970 interface with the plurality of access points such that a second passageway 960 is defined between the third access point 916c and the first access point 916a to provide fluid connection between the drug product port 940 and the infusion port 920. In this configuration, the second movable conduit 973 defines the second passageway 960. The second movable conduit 973 may be bent and/or may form an open space within the central interface member 910 that allows the drug product to pass between the third access point 916c and the first access point 916a. As described herein, the first end 941 of the drug product port 940 may be, or may be engaged with, the vial adaptor 980 configured to engage with the vial 994 such that the drug product flows from the vial and the vial adaptor 980, through the drug product port 940 and the second passageway 960 formed by the second movable conduit 973 to the infusion port 920.

Consistent with implementations of the current subject matter, the drug product delivery apparatus 900 may include a control member 990, as shown in FIG. 9A and FIG. 9D. The control member 990 is coupled to the movable conduits 970 to move the movable conduits 970 between the first position and the second position. For example, the control member 990 may be configured such that movement, such as rotation, of the control member 990 causes the movable conduits 970 to rotate within the cavity 912 of the central interface member 910. Thus, movement of the control member 990 causes the movable conduits 970 to be aligned to form the first passageway 950 and the second passageway 960.

In some implementations, the control member 990 is positioned on a first side of the central interface member 910. As shown in FIGS. 9A-9F, when facing towards the control member 990, the drug product port 940 is positioned to the left of the saline port 930. Such configurations may help to improve the ergonomics of the drug product delivery apparatus 900 and improve the experience of using the drug product delivery apparatus 900.

FIG. 9G illustrates aspects of operation of the drug product delivery apparatus 900, consistent with implementations of the current subject matter. Drug product delivery utilizing the drug product delivery apparatus 900 includes, according to some implementations, one or more stages, such as one, two, three, or more stages. For example, the stages of drug product delivery utilizing the drug product delivery apparatus 900 may include three stages. While this example is shown as having three stages, other number of stages may be performed, and in some instances, only one or two of the three illustrated stages are performed. As shown in FIG. 9G, drug product delivery utilizing the drug product delivery apparatus 900 may include: priming 997, drug product delivery 998, and flushing 999. The drug product delivery apparatus 900 is shown in cross-sectional views to illustrate aspects of the movable conduits 970 (e.g., the first movable conduit 971 defining the first passageway 950 and the second movable conduit 973 defining the second passageway 960). Front views are also provided to illustrate a corresponding position of the control member 990. Each of the stages is accomplished via pumping from an infusion pump that interfaces with the IV administration set coupled to the infusion port 920. Additionally or alternatively, the infusion pump may cause (e.g., automatically, after receiving an input, and/or the like) the control member 990 to move the movable conduits 970 from the first position to the second position and/or from the second position to the first position.

In the priming stage 997, the movable conduits 970 are positioned in the first position such that the first passageway 950 is formed between the saline port 930 and the infusion port 920, allowing saline to flow from the saline source (e.g., a saline bag), through the first passageway 950, to the infusion port 920 (coupled to an IV administration set). The priming 997 involves infusing, via the saline port 930, a first quantity of saline into the drug product delivery apparatus 900. The saline port 930 may be terminated with a spike or the like for coupling to the saline source, such as the saline bag. The priming provides for the first quantity of saline to be distributed to the infusion port 920 through the first passageway 950.

For the stage of the drug product delivery 998, the movable conduits 970 are moved into the second position. For example, the control member 990 is rotated such that the second passageway 960 is formed between the drug product port 940 and the infusion port 920. This allows drug product to flow from the vial to the drug product port 940, through the second passageway 960, to the infusion port 920 (coupled to the IV administration set).

After the drug product delivery 998, the flushing 999 is the next stage of operation of the drug product delivery apparatus 900. The movable conduits 970 are returned to the first position such that the first passageway 950 is formed. A second quantity of saline is flushed through the first passageway 950 to the infusion port 920. The infusion of the second quantity of saline may ensure that the infusion volume of the drug product is delivered to the patient.

Thus, the drug product delivery apparatus 900 provides a more convenient and faster IV administration option for healthcare systems while improving patient experience and safety of the patient.

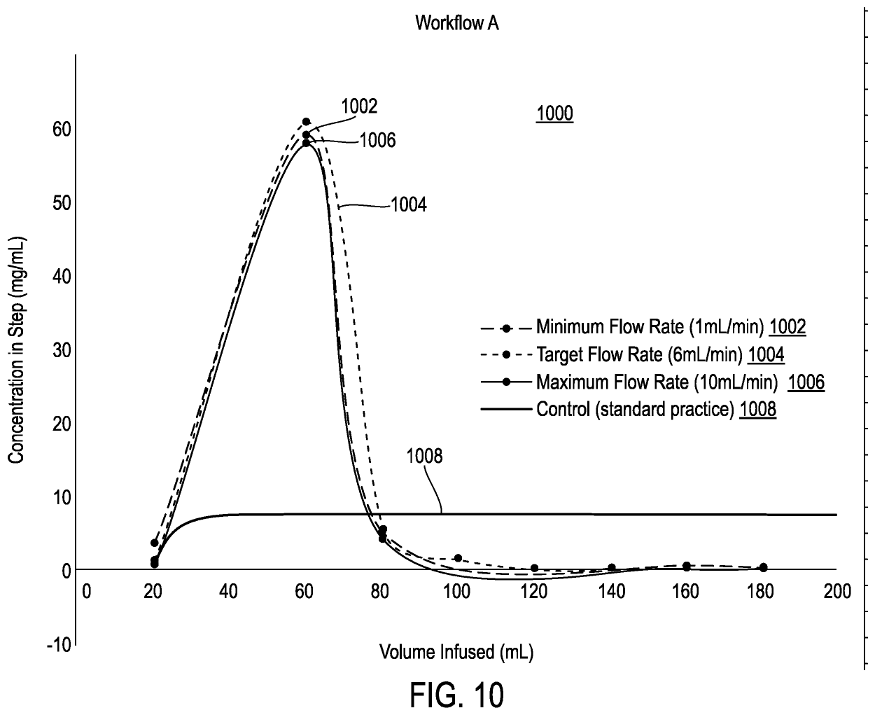

FIG. 10 depicts an example graph 1000 showing concentration kinetics during delivery of a drug product using the drug product delivery apparatus 900, consistent with implementations of the current subject matter. In other words, the graph 1000 depicts a comparison of protein concentration in the drug product and a volume of the drug product infused. In this example, concentration kinetics were recorded for delivery of 20 mL of a drug product. The drug product used was Tiragolumab with a protein concentration of 120 mg/mL. For example, the concentration of the drug product based on a volume of fluid infused via an IV administration set, such as the IV administration set 996, was compared across various flow rates, including a minimum flow rate (1 mL/min) 1002, a target flow rate (6 mL/min) 1004, a maximum flow rate (10 mL/min) 1006, and a control flow rate 1008. As shown on the graph 1000, protein concentration of the drug product within a collected fluid solution (including saline and/or the drug product) was measured at eight data collection points for each flow rate—(1) after 20 mL of the fluid solution was collected; (2) after 60 mL of the fluid solution was collected; (3) after 80 mL of the fluid solution was collected; (4) after 100 mL of the fluid solution was collected; (5) after 120 mL of the fluid solution was collected; (6) after 140 mL of the fluid solution was collected; (7) after 160 mL of the fluid solution was collected; and (8) after 180 mL of the fluid solution was collected.

The graph 1000 shows that delivery of the desired concentration of drug product using the drug product delivery apparatus 1100 (or any of the drug product delivery apparatuses described herein) can beneficially occur with less volume of the drug product and therefore in less time. Thus, such implementations may be more convenient for patients, as patients would spend less time in their chairs, less time waiting for a chair to open up, and less time waiting for preparation of the drug product. Such configurations may also allow nurses or other medical professionals to spend less time managing each patient and/or may ease pharmacy workloads.

During the testing of the drug product concentration using each flow rate (e.g., the minimum flow rate (1 mL/min) 1002, the target flow rate (6 mL/min) 1004, the maximum flow rate (10 mL/min) 1006, and the control flow rate 1008), the method shown in FIG. 9G was implemented, including the priming 997, drug product delivery 998, and flushing 999 stages.

First, the priming stage 997 involved infusing, via the saline port 930, a first quantity of saline into the drug product delivery apparatus 900. During the infusing of the first quantity of saline, the movable conduits 970 were positioned in the first position such that the first passageway 950 was formed between the saline port 930 and the infusion port 920, allowing saline to flow from the saline source (e.g., a saline bag), through the first passageway 950, to the infusion port 920 (coupled to an IV administration set). After the priming stage 997, at least some saline remained within the drug product delivery apparatus 900.

After the priming 997, the control member 990 was rotated such that the second passageway 960 is formed between the drug product port 940 and the infusion port 920. This allows drug product to flow from the vial to the drug product port 940, through the second passageway 960, to the infusion port 920 (coupled to the IV administration set). Thus, the movable conduits 970 were moved into the second position.

The infusion pump coupled to the drug product delivery apparatus 900 was switched ON and the flow rate was set to either the minimum flow rate (1 mL/min) 1002, the target flow rate (6 mL/min) 1004, the maximum flow rate (10 mL/min) 1006, or the control flow rate 1008, depending on the flow rate being tested.

Next, during the drug product delivery 998, the drug product was withdrawn from the drug product source (e.g., the vial) by the drug product delivery apparatus 900 and a fluid solution of the fluid drawn through the drug product delivery apparatus 900 was collected in a beaker. The fluid solution included saline and drug product. At each data collection point (e.g., at 20 mL, 60 mL, 80 mL, 100 mL, 120 mL, 140 mL, 160 mL, and 180 mL of solution collected), the protein concentration of the collected solution was measured. At each data collection point, the infusion pump automatically stopped operation (or switched OFF) to allow for measurement of the protein concentration. As shown in the graph 1000, at the first data collection point (e.g., at 20 mL of fluid solution collected), the protein concentration was low, since most of the collected fluid solution included the saline present in the tubing as a result of the priming 997. After the first data collection point (e.g., at 20 mL of fluid solution collected) the drug product source had been emptied.

Next, at the flushing stage 999, the control member 990 was rotated such that the movable conduits 970 were returned to the first position to form the first passageway 950. The infusion pump was switched ON to continue drawing the fluid solution through the drug product delivery apparatus 900 to be collected at the beaker. After the first data collection point, an additional 40 mL of fluid solution was collected by the beaker and the infusion pump automatically stopped operation for the protein concentration of the collected fluid solution to be measured. At the second data collection point, (e.g., at 60 mL of fluid solution collected), the protein concentration is higher than at the first data collection point because most of the collected fluid solution at this point included the drug product. The flushing stage 999 continued in 20 mL increments until a total of 180 mL of the fluid solution was collected.

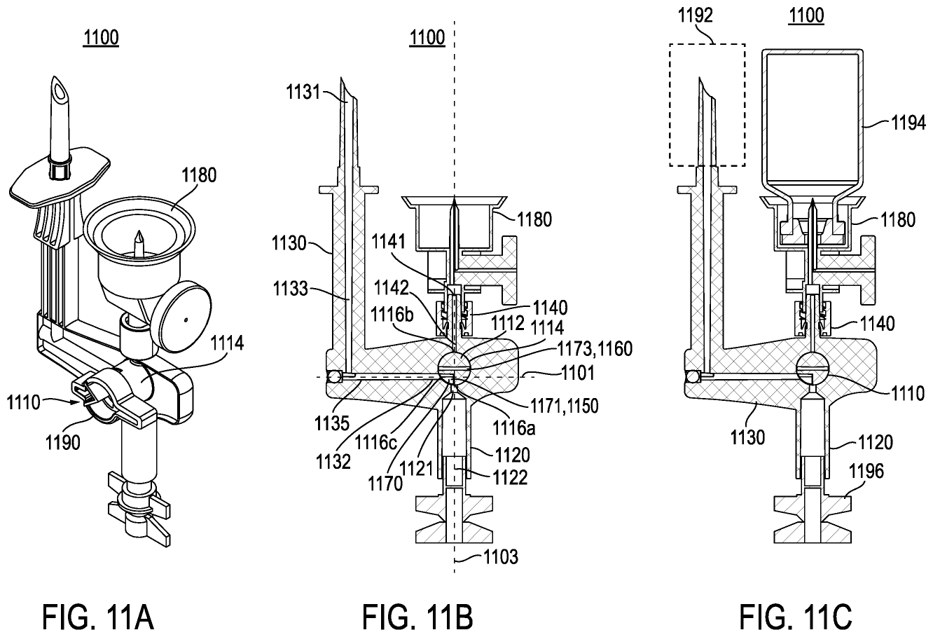

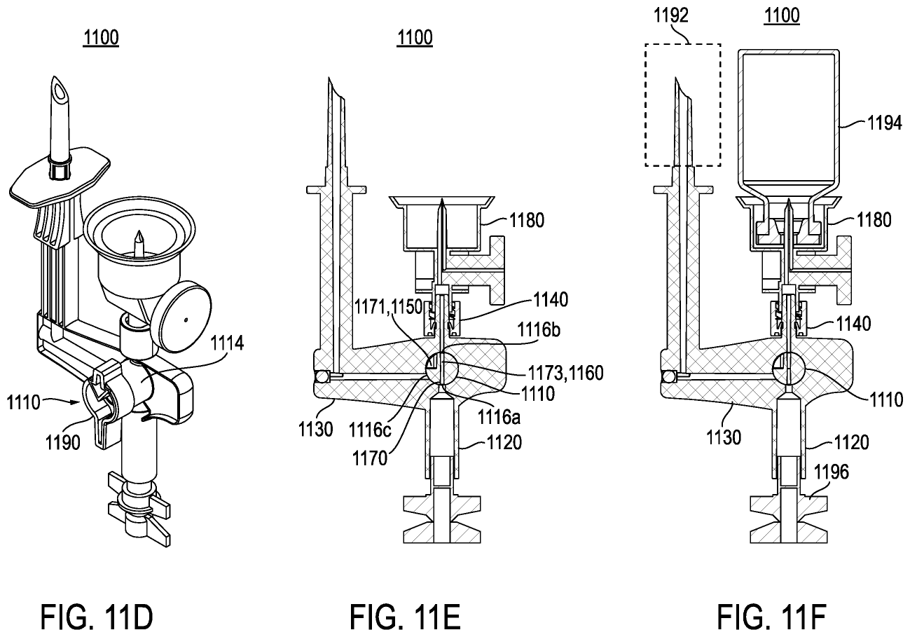

FIG. 11A-FIG. 11G illustrate aspects of a drug product delivery apparatus 1100 consistent with implementations of the current subject matter. FIG. 11A is a perspective view of the drug delivery apparatus 1100 in a first position and FIG. 11B and FIG. 11C are cross-sectional views of the drug product delivery apparatus 1100 in the first position. FIG. 11D is a perspective view of the drug delivery apparatus 1100 in a second position and FIG. 11E and FIG. 11F are cross-sectional views of the drug product delivery apparatus 1100 in the second position. FIG. 11C and FIG. 11F show the drug product delivery apparatus 1100 coupled to a saline source 1192 and a vial 1194 containing a drug product.

The drug product delivery apparatus 1100 includes a central interface member 1110. The central interface member 1110 includes a cavity 1112 surrounded by an outer wall 1114 and a plurality of access points formed through respective surfaces of the outer wall 1114. For example, the plurality of access points may include one, two, three, four, or more access points. Referring to FIGS. 11B, 11C, 11E, and 11F, the central interface member 1110 may include at least three access points, such as a first access point 1116a, a second access point 1116b, and a third access point 1116c. The access points may provide access to the cavity 1112, and may allow for a fluid to enter and/or exit the cavity 1112 of the central interface member 1110.

The drug product delivery apparatus 1100 also includes an infusion port 1120, a saline port 1130, and a drug product port 1140. According to aspects of the current subject matter, each of the ports 1120, 1130, and 1140 has a first end and a second end, and each of the ports 1120, 1130, and 1140 is coupled at one end to a respective access point 1116a, 1116b, and 1116c of the central interface member 1110.

The infusion port 1120 has a first end 1121, coupled to the first access point 1116a of the central interface member 1110, and a second end 1122. The second end 1122 may be opposite the first end 1121. The second end 1122 of the infusion port 1120 may connect to an IV administration set 1196 for delivering fluid from the infusion port 1120. For example, the fluid passing through the cavity 1112 of the central interface member 1110 may be delivered to the IV administration set 1196 via the infusion port 1120. The infusion port 1120 may be coupled to a luer lock or other connector, which is coupled to tubing or another delivery mechanism for delivering the fluid to the patient.

The saline port 1130 of the drug product delivery apparatus 1100 has a first end 1131 and a second end 1132. The second end 1132 of the saline port 1130 is coupled to the third access point 1116c of the central interface member 1110. In some implementations, the second end 1132 and the first end 1131 are positioned along a single axis.

In some implementations, such as in the configuration shown in FIGS. 11A-11F, the first end 1131 is positioned in a direction that is perpendicular relative to the second end 1132. For example, the saline port 1130 may include a first saline passageway 1133 and a second saline passageway 1135. The first saline passageway 1133 and the second saline passageway 1135 may be fluidly coupled and/or integrally formed to define the saline port 1130. The first saline passageway 1133 may extend between the first end 1131 and the second saline passageway 1135. The second saline passageway 1135 may extend between the first saline passageway 1133 and the second end 1132. The second saline passageway 1135 may extend laterally from the central interface member 1110, such as along a lateral axis 1101 of the central interface member 1110 that is perpendicular to the longitudinal axis 1103 of the central interface member 1110. The first saline passageway 1133 may extend in a direction that is perpendicular relative to the lateral axis 1101 and/or to the second saline passageway 1135, though other angles are contemplated consistent with implementations of the current subject matter.

Referring again to FIGS. 11A-11F, the first end 1131 of the saline port 1130 is configured to be coupled to a saline source. Thus, saline from the saline source may pass through the saline port 1130, into and through the cavity 1112, and through the infusion port 1120 to be delivered to the IV administration set 1196 to prime and/or flush the IV administration set 1196. In some implementations, the saline port 1130 is terminated with a spike for coupling to the saline source (e.g., a saline bag or the like).

The drug product port 1140 has a first end 1141 and a second end 1142. The second end 1142 may be opposite the first end 1141. The second end 1142 of the drug product port 1140 is coupled to the second access point 1116b of the central interface member 1110. The first end 1141 of the drug product port 1140 is configured to be coupled to a drug product source, thus allowing the drug product port 1140 to deliver drug product from the drug product source through the second passageway 1160 to the infusion port 1120. The first end 1141 of the drug product port 1140 may be, or may be engaged with, a vial adaptor 1180 configured to engage with the vial 1194 (shown in FIG. 11C and FIG. 11F) such that the drug product flows from the vial and the vial adaptor 1180, through the drug product port 1140, into the cavity 1112 of the central interface member 1110, and to the infusion port 1120.

The drug product port 1140 may be vertically aligned with the infusion port 1120. For example, the drug product port 1140 and the infusion port 1120 may be aligned along a central longitudinal axis 1103 of the central interface member 1110. In some implementations, the drug product port 1140 and the infusion port 1120 are positioned directly opposite from one another across the central interface member 1110. Such configurations allow for the saline to easily flow between the drug product port 1140 and the infusion port 1120. Such configurations may additionally or alternatively reduce a volume of drug product that remains within the drug product port 1140 after the drug product is delivered to the IV administration set 1196. For example, in some instances when the drug product port 1140 includes a bend and/or otherwise includes multiple passageways, a small volume of the drug product may remain within the drug product port 1140 after the drug product is delivered to the IV administration set for delivery to the patient. The drug product delivery apparatus 1100 consistent with implementations of the current subject matter may help to reduce or eliminate the volume of drug product remaining within the drug product port 1140 or another portion of the drug product delivery apparatus 1100. For example, since the drug product port 1140 may not include a bend and an entirety of the passageway of the drug product port 1140 may be longitudinally aligned, along the longitudinal axis 1103, with the infusion port 1120, the drug product may easily flow between the drug product port 1140 and the infusion port 1120. Thus, the drug product delivery apparatus 1100 may reduce an amount of drug product that is wasted, and may help to ensure that the proper amount of drug product is delivered to the patient.

The drug product delivery apparatus 1100 may include movable conduits 1170, as shown in the cross-sectional views of FIG. 11B, FIG. 11C, FIG. 11E, and FIG. 11F. The movable conduits 1170 are positioned within the cavity 1112 and interface with the plurality of access points, such as the first, second, and third access points 1116a, 1116b, 1116c. The movable conduits 1170 may include one, two, three, four, five, or more movable conduits 1170. For example, as shown in FIGS. 11B, 11C, 11E, and 11F, the movable conduits 1170 include a first movable conduit 1171 and a second movable conduit 1173. Each of the movable conduits 1170 may be separated from one another such that each of the movable conduits are not in fluid communication with one another. In other implementations, one or more of the movable conduits 1170 may be fluidly coupled with one another.

Consistent with implementations of the current subject matter, the movable conduits 1170 are configured to move between at least a first position (see FIGS. 11A-1C) and a second position (see FIGS. 11D-11F). In the first position, saline is permitted to pass through the central interface member 1110, such as from the saline source 1192 to the IV administration set 1196. In the first position, the drug product may not be permitted to pass through the central interface member 1110. For example, in the first position, openings providing access to the second movable conduit 1173 may not be fluidly connected with the first, second, and/or third access point 1116a, 1116b, 1116c. Instead, the openings into the second movable conduit 1173 may be blocked by the outer wall 1114 surrounding the cavity 1112.

In the second position, the drug product is permitted to pass through the central interface member 1110, such as from the vial 1194 to the IV administration set 1196. In the second position, the saline may not be permitted to pass through the central interface member 1110. For example, in the second position, openings providing access to the first movable conduit 1173 may not be fluidly connected with the first, second, and/or third access point 1116a, 1116b, 1116c. Instead, the openings into the first movable conduit 1171 may be blocked by the outer wall 1114 surrounding the cavity 1112.

In other words, the first position of the movable conduits and the second position of the movable conduits 1170 do not concurrently occur. That is, while a first passageway 1150 is formed and open between the third access point 1116c and the first access point 1116a to provide fluid connection between the saline port 1130 and the infusion port 1120, a second passageway 1160 is not formed (e.g., the movable conduits 1170 are positioned in such a way that there is no fluid connection between the drug product port 1140 and the infusion port 1120). When the second passageway 1160 is formed and open between the second access point 1116b and the first access point 1116a to provide fluid connection between the drug product port 1140 and the infusion port 1120, the first passageway 1150 is not formed (e.g., the movable conduits 1170 are positioned in such a way that there is no fluid connection between the saline port 1130 and the infusion port 1120). This arrangement allows for saline priming and flushing to occur between the saline port 1130 and the infusion port 1120 separate from delivery of the drug product from the drug product port 1140 to the infusion port 1120.

Consistent with implementations of the current subject matter, in the first position, the movable conduits 1170 interface with the plurality of access points such that a first passageway 1150 is defined between the third access point 1116c and the first access point 1116a to provide fluid connection between the saline port 1130 and the infusion port 1120. In this configuration, the first movable conduit 1171 defines the first passageway 1150. The first movable conduit 1171 may be bent and/or may form an open space within the central interface member 1110 that allows the saline to pass between the third access point 1116c and the first access point 1116a. As noted above, the first end 1131 of the saline port 1130 is configured to be coupled to a saline source, thus allowing the saline port 1130 to deliver saline from the saline source through the first passageway 1150 formed by the first movable conduit 1171 to the infusion port 1120.

In the second position, the movable conduits 1170 interface with the plurality of access points such that a second passageway 1160 is defined between the third access point 1116b and the first access point 1116a to provide fluid connection between the drug product port 1140 and the infusion port 1120. In this configuration, the second movable conduit 1173 defines the second passageway 1160. The second movable conduit 1173 may form a straight (e.g., unbent) channel that extends between the first access point 1116a and the second access point 1116b. As described herein, the first end 1141 of the drug product port 1140 may be, or may be engaged with, the vial adaptor 1180 configured to engage with the vial 1194 such that the drug product flows from the vial and the vial adaptor 1180, through the drug product port 1140 and the second passageway 1160 formed by the second movable conduit 1173 to the infusion port 1120.

Consistent with implementations of the current subject matter, the drug product delivery apparatus 1100 may include a control member 1190, as shown in FIG. 11A and FIG. 11D. The control member 1190 is coupled to the movable conduits 1170 to move the movable conduits 1170 between the first position and the second position. For example, the control member 1190 may be configured such that movement, such as rotation, of the control member 1190 causes the movable conduits 1170 to rotate within the cavity 1112 of the central interface member 1110. Thus, movement of the control member 1190 causes the movable conduits 1170 to be aligned to form the first passageway 1150 and the second passageway 1160.

In some implementations, the control member 1190 is positioned on a first side of the central interface member 1110. As shown in FIGS. 11A-11F, when facing towards the control member 1190, the saline port 1130 is positioned to the left of the drug product port 1140. Such configurations may help to improve the ergonomics of the drug product delivery apparatus 1100 and improve the experience of using the drug product delivery apparatus 1100.

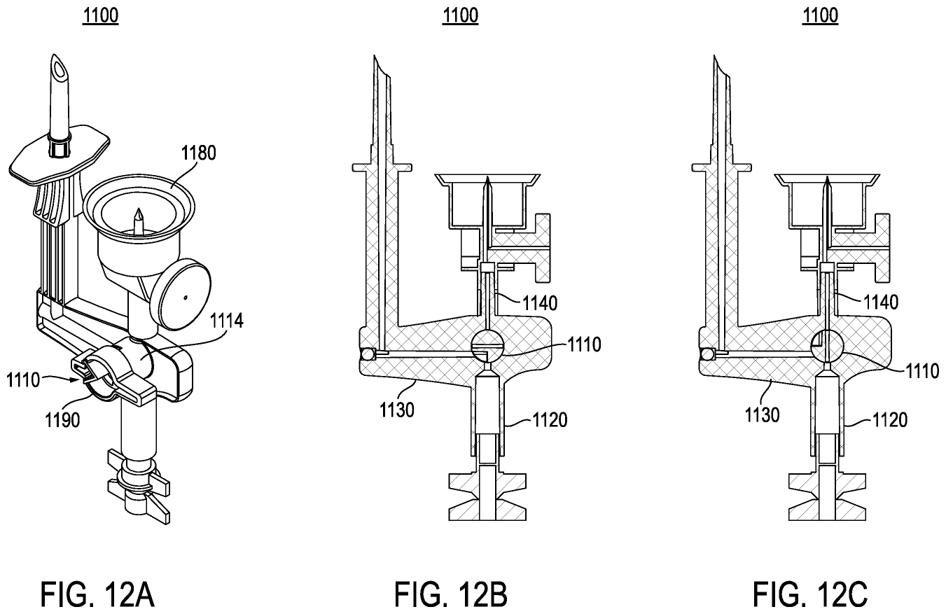

In some implementations, as shown in FIGS. 11A-11F, the drug product port 1140 is coupled to the central interface member 1110 via a locking nut. FIG. 12A, FIG. 12B, and FIG. 12C illustrate another example of the drug product delivery apparatus 1110 in which the drug product port 1140 is directly coupled to the central interface member 1110. For example, the drug product port 1140 may be integrally formed with the central interface member 1110, the drug product port 1140 and the central interface member 1110 may be molded as a single component, and/or the drug product port 1140 may be bonded (e.g., solvent bonded) to the central interface member 1110. Such configurations may help to reduce an amount of drug product that remains within the drug product port 1140 after the drug product is delivered to the IV administration set, such as via the infusion port 1120.

Consistent with implementations of the current subject matter, drug product delivery utilizing the drug product delivery apparatus 1100 includes, according to some implementations, one or more stages, such as one, two, three, or more stages. For example, the stages of drug product delivery utilizing the drug product delivery apparatus 1100 may include three stages. While this example is described as having three stages, other number of stages may be performed, and in some instances, only one or two of the three stages are performed. For example, drug product delivery utilizing the drug product delivery apparatus 1100 may include: priming, drug product delivery, and flushing. Each of the stages is accomplished via pumping from an infusion pump that interfaces with the IV administration set coupled to the infusion port 1120. Additionally or alternatively, the infusion pump may cause (e.g., automatically, after receiving an input, and/or the like) the control member 1190 to move the movable conduits 1170 from the first position to the second position and/or from the second position to the first position.

In the priming stage, the movable conduits 1170 are positioned in the first position such that the first passageway 1150 is formed between the saline port 1130 and the infusion port 1120, allowing saline to flow from the saline source (e.g., a saline bag), through the first passageway 1150, to the infusion port 1120 (coupled to an IV administration set). The priming 1197 involves infusing, via the saline port 1130, a first quantity of saline into the drug product delivery apparatus 1100. The saline port 1130 may be terminated with a spike or the like for coupling to the saline source, such as the saline bag. The priming provides for the first quantity of saline to be distributed to the infusion port 1120 through the first passageway 1150.

For the stage of the drug product delivery, the movable conduits 1170 are moved into the second position. For example, the control member 1190 is rotated such that the second passageway 1160 is formed between the drug product port 1140 and the infusion port 1120. This allows drug product to flow from the vial to the drug product port 1140, through the second passageway 1160, to the infusion port 1120 (coupled to the IV administration set).

After the drug product delivery, the flushing is the next stage of operation of the drug product delivery apparatus 1100. The movable conduits 1170 are returned to the first position such that the first passageway 1150 is formed. A second quantity of saline is flushed through the first passageway 1150 to the infusion port 1120. The infusion of the second quantity of saline may ensure that the infusion volume of the drug product is delivered to the patient.

Thus, the drug product delivery apparatus 1100 provides a more convenient and faster IV administration option for healthcare systems while improving patient experience and safety of the patient.

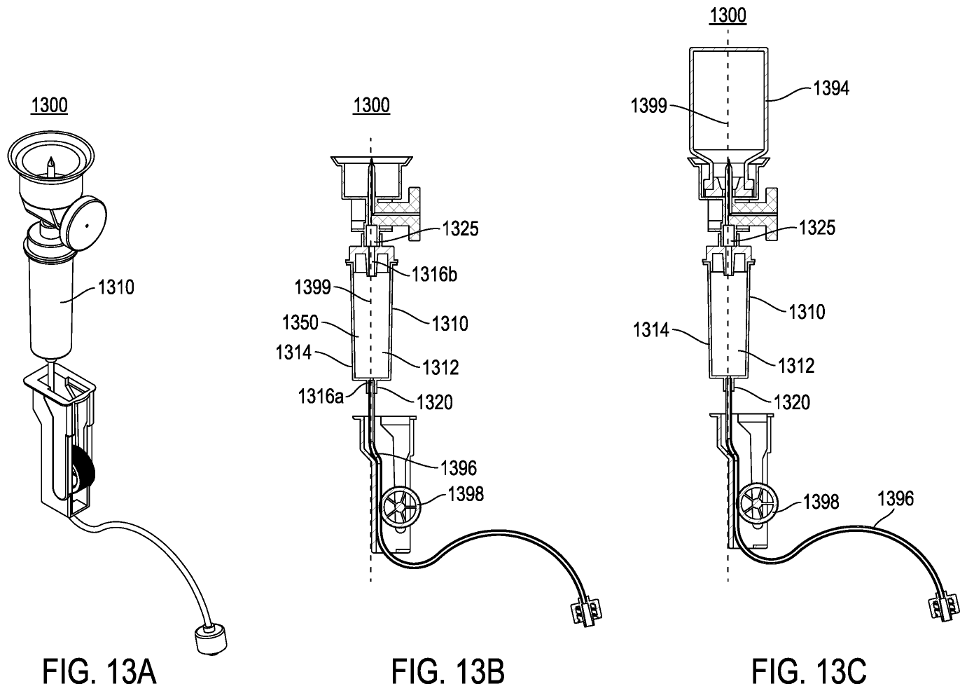

FIG. 13A-FIG. 13C illustrate aspects of a drug product delivery apparatus 1300 consistent with implementations of the current subject matter. FIG. 13A is a perspective view of the drug delivery apparatus 1300, FIG. 13B is a cross-sectional view of the drug product delivery apparatus 1300, and FIG. 13C is a cross-sectional view of the drug product delivery apparatus 1300 coupled to a vial 1394 containing a drug product.

The drug product delivery apparatus 1300 includes a central interface member 1310. The central interface member 1310 includes a cavity 1312 surrounded by an outer wall 1314 and a plurality of access points 1316 formed through respective surfaces of the outer wall 1314. The drug product delivery apparatus 1300 may include two access points: a first access point 1316a and a second access point 1316b.

The drug product delivery apparatus 1300 also includes an infusion port 1320 and a fluid port 1325. The fluid port 1325 may define both a saline port and a drug product port. However, the fluid port 1325 may define only the drug product port, the drug product delivery apparatus 1300 may not use saline. For example, the drug product delivery apparatus 1300 may beneficially allow for infusion of the appropriate amount of drug product without the use of saline for flushing the drug product delivery apparatus 1300. Each of the ports 1320, 1325 is coupled to and/or defines a respective access point 1316a, 1316b of the central interface member 1310. For example, the infusion port 1320 may be coupled to and/or define the first access point 1316a of the central interface member 1310. In other words, the infusion port 1320 may be an opening of the central interface member 1310.

The first access point 1316a and the second access point 1316b may be aligned along a central longitudinal axis 1399of the central interface member 1310. The first access point 1316a and the second access point 1316b may also be aligned with a center of the fluid port 1325 and/or a center of the infusion port 1320. Thus, in some implementations, the first access point 1316a, the second access point 1316b, the fluid port 1325, and the infusion port 1320 may be aligned along the central longitudinal axis 1399of the central interface member 1310. Such configurations may help to more quickly deliver the drug product through the drug product delivery apparatus 1300, while minimizing an amount of the drug product remaining within the drug product delivery apparatus 1300 after infusion. In some implementations, a center of the drug product source, such as the vial is also aligned along the central longitudinal axis 1399.

In some implementations, the drug product delivery apparatus 1300 serves as an IV administration set such that no additional IV administration set is coupled to the drug product delivery apparatus. Instead, the drug product delivery apparatus 1300 may deliver the drug product directly to the patient via tubing 1396. In other implementations, the infusion port 1320 may be configured to connect to an IV administration set for delivering fluid from the infusion port 120. In some implementations, a flow stop 1398 or other mechanism controls the flow of fluid from within the cavity 1312 to the patient (or to a separate IV administration set) through the tubing 1396. The tubing 1396 may include various lengths, ranging from several inches to several feet, depending on the implementation.

The fluid port 1325 may be coupled to and/or may define the second access point 1316b of the central interface member 1310. A first passageway 1350 is thus formed between the fluid port 1325 and the infusion port 1320 by way of the second access point 116b and the first access point 116a. The first passageway 1350 is a fluidic connection through the cavity 1312 of the central interface member 1310.

The fluid port 1325 is configured to be coupled to a drug product source, thus allowing the fluid port 1325 to deliver drug product from the drug product source through the cavity 1312 of the central interface member 1310 (e.g., through the first passageway 1350) to the infusion port 1320. The drug product source may be, for example, a syringe. The drug product source may be, for example, a vial or a container. In some implementations, the fluid port 1325 may be, or may be engaged with, a vial adaptor configured to engage with the vial such that the drug product flows from the vial and the vial adaptor, through the fluid port 1325, into the cavity 1312 of the central interface member 1310. As a result, the fluid port 1325 may be a single port that is configured to interface with one or more drug product sources. In other implementations, the fluid port is configured to be non-contemporaneously coupled to a saline source and a drug product source.

As noted above, in some implementations, the drug product delivery apparatus 1300 may not be used with saline to prime and/or flush the drug product delivery apparatus 1300. Thus, drug product delivery utilizing the drug product delivery apparatus 1300 includes, according to some implementations, two stages: drug product withdrawal and drug product administration. While this example is described as having two stages, other number of stages (e.g., one, two, three, four, five, or more) may be performed. For example, in some instances in which the drug product delivery apparatus 1300 is used with saline, the drug product delivery apparatus 1300 may be primed before the drug product withdrawal and/or the drug product administration, and/or the drug product delivery apparatus 1300 may be flushed after the drug product withdrawal and/or the drug product administration.

The drug product withdrawal, consistent with implementations of the current subject matter, may include withdrawing, with a syringe or the like, a quantity (e.g., an infusion volume) of the drug product from the drug product source, such as a vial or container. In other implementations, the drug product source may be coupled directly to the fluid port 1325, and the drug product may be withdrawn from the drug product source.

The drug product injection includes injecting the infusion volume of the drug product, contained in the syringe and/or the drug product source, such as the vial, via the fluid port 1325. For example, the infusion volume may be injected through the fluid port 1325 to the cavity 1312, and into the tubing 1396 for delivery to the patient.

### V. Methods of Administration

In an aspect, a method of intravenously administering a drug product to a patient is provided. The method may utilize a point of care drug product delivery apparatus as described herein.

The method may include priming the infusion line with a first quantity of saline (or other appropriate liquid). For example, a first quantity of saline may be provided in a saline bag or container (containing, for example, 0.9% NaCl), and introduced (e.g., via the pump operation) into the infusion line. This may be done so that air is removed from the infusion line prior to the drug infusion into the patient (and prior to the IV catheter insertion in the patient).

The drug product may be administered using an infusion pump. Upon completion of the infusion volume of the drug product being administered to the patient, a second drug product may be administered to the patient following the same or similar procedure. The second drug product may be of the same type or a different type as the drug product that was first administered. Upon completion of the infusion volume of the drug product being administered or following the administration of the second drug product, the infusion line may be flushed with a second quantity of saline.

A process for administering a drug to a patient, consistent with additional implementations of the current subject matter, may include infusing into the patient via an infusion line a first quantity of saline, followed by infusing into the patient via the infusion line an infusion volume of the drug product for a first period of time. The drug product may be administered at a fixed dose (e.g., the same dose regardless of the patient age and/or weight) or at a weight-based dose.

An initial amount of drug product in the vial may be less than or equal to about 100 mL, about 90 mL, about 80 mL, about 70 mL, about 60 mL, about 50 mL, about 40 mL, about 30 mL, about 20 mL, about 10 mL, or about 5 mL. In some embodiments, the initial amount of drug in the vial is between about 1 mL and about 30 mL. In some embodiments, the initial amount of drug in the vial is between about 1 mL and about 20 mL. In some embodiments, the initial amount of drug in the vial is between about 1 mL and about 15 mL. In some embodiments, the initial amount of drug in the vial is between about 5 mL and about 30 mL. In some embodiments, the initial amount of drug in the vial is between about 10 mL and about 30 mL. In some embodiments, the initial amount of drug in the vial is between about 15 mL and about 30 mL. In some embodiments, the initial amount of drug in the vial is between about 5 mL and about 25 mL. In some embodiments, the initial amount of drug in the vial is between about 5 mL and about 20 mL. In some embodiments, the initial amount of drug in the vial is between about 5 mL and about 15 mL. The amount may be any value or subrange within the recited ranges, including endpoints.

In some embodiments, the initial amount of drug in the vial is less than or equal to about 30 mL, about 29 mL, about 28 mL, about 27 mL, about 26 mL, about 25 mL, about 24 mL, about 22 mL, or about 21 mL. In some embodiments, the initial amount of drug in the vial is less than or equal to about 20 mL. In some embodiments, the initial amount of drug in the vial is less than or equal to about 19 mL. In some embodiments, the initial amount of drug in the vial is less than or equal to about 18 mL. In some embodiments, the initial amount of drug in the vial is less than or equal to about 17 mL. In some embodiments, the initial amount of drug in the vial is less than or equal to about 16 mL. In some embodiments, the initial amount of drug in the vial is less than or equal to about 15 mL. In some embodiments, the initial amount of drug in the vial is less than or equal to about 14 mL. In some embodiments, the initial amount of drug in the vial is less than or equal to about 13 mL. In some embodiments, the initial amount of drug in the vial is less than or equal to about 12 mL. In some embodiments, the initial amount of drug in the vial is less than or equal to about 11 mL. In some instances, the initial amount of drug in the vial is less than or equal to about 10 mL. In some embodiments, the initial amount of drug in the vial is less than or equal to about 5 mL.

The infusion volume of the drug may be between about 10 mL and about 100 mL, and the second quantity of saline may be between about 25 mL and about 90 mL.

In embodiments, the second quantity of saline may be between about 20 mL and about 100 mL. In embodiments, the second quantity of saline may be between about 25 mL and about 90 mL. In embodiments, the second quantity of saline may be between about 25 mL and about 80 mL. In embodiments, the second quantity of saline may be between about 25 mL and about 70 mL. In embodiments, the second quantity of saline may be between about 25 mL and about 60 mL. In embodiments, the second quantity of saline may be between about 25 mL and about 50 mL. In embodiments, the second quantity of saline may be between about 25 mL and about 40 mL. In embodiments, the second quantity of saline may be between about 25 mL and about 30 mL. The amount may be any value or subrange within the recited ranges, including endpoints.

The drug product and/or second quantity of saline may be infused into the patient at an infusion rate between about 1 mL/min and about 10 mL/min. The drug product and/or second quantity of saline may be infused into the patient at an infusion rate between about 2 mL/min and about 10 mL/min. The drug product and/or second quantity of saline may be infused into the patient at an infusion rate between about 3 mL/min and about 10 mL/min. The drug product and/or second quantity of saline may be infused into the patient at an infusion rate between about 1 mL/min and about 8 mL/min. The drug product and/or second quantity of saline may be infused into the patient at an infusion rate between about 1 mL/min and about 6 mL/min. The drug product and/or second quantity of saline may be infused into the patient at an infusion rate of about 1 mL/min. The drug product and/or second quantity of saline may be infused into the patient at an infusion rate of about 2 mL/min. The drug product and/or second quantity of saline may be infused into the patient at an infusion rate of about 3 mL/min. The drug product and/or second quantity of saline may be infused into the patient at an infusion rate of about 4 mL/min. The drug product and/or second quantity of saline may be infused into the patient at an infusion rate of about 5 mL/min. The drug product and/or second quantity of saline may be infused into the patient at an infusion rate of about 6 mL/min. The drug product and/or second quantity of saline may be infused into the patient at an infusion rate of about 7 mL/min. The drug product and/or second quantity of saline may be infused into the patient at an infusion rate of about 8 mL/min. The drug product and/or second quantity of saline may be infused into the patient at an infusion rate of about 9 mL/min. The drug product and/or second quantity of saline may be infused into the patient at an infusion rate of about 10 mL/min. The amount may be any value or subrange within the recited ranges, greater than the recited ranges, and/or less than the recited ranges, including endpoints.

Although the disclosure, including the figures, described herein may describe and/or exemplify different variations separately, it should be understood that all or some, or components of them, may be combined.

Although various illustrative embodiments are described above, any of a number of changes may be made to various embodiments. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the claims.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. References to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as, for example, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings provided herein.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” “or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. In embodiments, “about” refers to +/−10% or less of the stated value (or range of values). Any numerical values given herein should also be understood to include about or approximately that value, unless the context indicates otherwise.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, are possible.

In the descriptions above and in the claims, phrases such as, for example, “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

What is claimed is:

1. An apparatus, comprising:
a central interface member comprising a cavity surrounded by an outer wall, a plurality of access points formed through respective surfaces of the outer wall;
an infusion port comprising a first end and a second end, the first end of the infusion port coupled to a first access point of the plurality of access points;
a saline port comprising a first end and a second end, the second end of the saline port coupled to a second access point of the plurality of access points, such that a first passageway is formed between the saline port and the infusion port through the cavity of the central interface member; and
a drug product port comprising a first end and a second end, the second end of the drug product port coupled to a third access point of the plurality of access points, such that a second passageway is formed between the drug product port and the infusion port through the cavity of the central interface member.

2. The apparatus of claim 1, wherein the central interface member, the infusion port, the saline port, and the drug product port comprise a single molded component.

3. The apparatus of claim 1, wherein the second end of the infusion port is configured to connect to an intravenous administration set.

4. The apparatus of claim 3, wherein the infusion port is configured to deliver saline and drug product to a patient via the intravenous administration set.

5. The apparatus of claim 1, wherein the first end of the saline port is configured to be coupled to a saline source, wherein the saline port is configured to deliver saline from the saline source through the cavity of the central interface member to the infusion port.

6. The apparatus of claim 5, wherein the saline port is terminated with a spike.

7. The apparatus of claim 1, wherein the first end of the drug product port is configured to be coupled to a drug product source, wherein the drug product port is configured to deliver drug product from the drug product source through the cavity of the central interface member to the infusion port.

8. The apparatus of claim 1, further comprising:
a second central interface member comprising a second cavity surrounded by a second outer wall, a plurality of second access points formed through respective surfaces of the second outer wall;
a second infusion port comprising a first end and a second end, the first end of the second infusion port coupled to a first access point of the plurality of second access points;
a second saline port comprising a first end and a second end, the second end of the second saline port coupled to a second access point of the plurality of second access points, the first end of the second saline port coupled to the second end of the infusion port to fluidly connect the infusion port and the second saline port; and
a second drug product port comprising a first end and a second end, the second end of the second drug product port coupled to a third access point of the plurality of second access points to fluidly connect the second drug product port and the second infusion port through the second cavity of the second central interface member.

9. The apparatus of claim 7, wherein the drug product source comprises a syringe.

10. The apparatus of claim 7, wherein the drug product source comprises at least one vial, wherein the first end of the drug product port comprises at least one vial adaptor configured to engage with a respective one of the at least one vial, wherein the drug product flows from the at least one vial and the at least one vial adaptor into the cavity of the central interface member.

11. The apparatus of claim 1, wherein the central interface member comprises an expandable chamber.

12. The apparatus of claim 1, further comprising:
movable conduits contained within the cavity of the central interface member, the movable conduits configured to interface with the plurality of access points to form a plurality of passageways between the plurality of access points;
wherein the movable conduits are configured to move between at least a first position and a second position, wherein in the first position the movable conduits interface with the plurality of access points such that the first passageway is defined between the second access point and the first access point to provide fluid connection between the saline port and the infusion port, and wherein in the second position the movable conduits interface with the plurality of access points such that the second passageway is defined between the third access point and the first access point to provide fluid connection between the drug product port and the infusion port.

13. The apparatus of claim 12, further comprising a control member coupled to the movable conduits and configured to rotate the movable conduits between the first position and the second position.

14. The apparatus of claim 12, wherein the saline port is configured to deliver saline from a saline source through the first passageway to the infusion port.

15. The apparatus of claim 12, wherein the drug product source comprises a vial, wherein the first end of the drug product port comprises a vial adaptor, the vial adaptor configured to engage with the vial such that the drug product flows through the second passageway to the infusion port.

16. The apparatus of claim 12, further comprising:
a flush port comprising a first end and a second end, the second end of the flush port coupled to a fourth access point of the plurality of access points of the central interface member;
wherein in the second position, the movable conduits interface with the plurality of access points such that a third passageway is defined between the second access point and the fourth access point to provide fluid connection between the saline port and the flush port.

17. The apparatus of claim 16, wherein the first end of the flush port is configured to be coupled to a drug product source such that saline flows through the third passageway to the drug product source.

18. The apparatus of claim 17, wherein the drug product source comprises a chamber in which an infusion volume of a drug product is contained.

19. The apparatus of claim 18, wherein the first end of the flush port is coupled to an upper portion of the chamber, wherein the first end of the drug product port is coupled to a lower portion of the chamber.

20. The apparatus of claim 18, wherein the chamber is configured to be coupled to a vial from which the chamber receives the infusion volume of the drug product.

21-83. (canceled)
